SEQUENCE LISTING (12 sequences)

SEQ ID NO: 1 - protein, 156 aa, synthetic construct
MSSTNNQLTVTNNSGVAQINAKNSGLYTTVYDTKGKTTNQIQRTLSVTKAATLGDKKFYLVGDYNTGTNYGWVKQDEVIYNTAKSPVKINQTYNVKPGVKLHTVPWGTYNQVAGTVSGKGDQTFKATKQQQIDKATYLYGTVNGKSGWISKYYLTA

SEQ ID NO: 2 - DNA, 830 bp, synthetic construct
  source 1..830: mol_type = other DNA
ccatgggcagccatcaccatcaccatcacagcagcggcttagtgccacgagggtctagcatgcagtacaaattagttatctgtggtaaaacattgaaaggcgaaacaactactaaagctgttgatgctgaaactgcagaaaaagctttcaaacaatacgctaacgacaacggtgttgacggtgtttggacttacgacgatgcgactaagacctttacagttactgaaggaggtggcggtagcggtagcgcaggtagcgaagcagcaggttcagaaggttcagctggctcagaagcagctggcagcgaaggtggtggtggtagtgaaaatttgtacttccaatccgggtctcatatgagcagcaccaataatcagctgaccgttaccaataatagcggtgttgcacagattaatgcgaaaaatagcggtctgtataccaccgtgtatgataccaaaggtaaaaccaccaatcagattcagcgtaccctgagcgtgaccaaagcagcaaccctgggtgataaaaagttttatctggtgggtgattataacaccggcaccaattatggttgggttaaacaggatgaagtgatttacaataccgcaaaaagtccggtgaaaatcaaccagacctataatgttaaaccgggtgttaaactgcataccgttccgtggggtacatataatcaggttgcaggcaccgttagcggtaaaggtgatcagacctttaaagcaaccaaacagcagcagattgataaagccacctatctgtatggtacggtgaatggtaaaagcggttggatcagcaaatattacctgaccgcataaggatcc

SEQ ID NO: 3 - protein, 56 aa, synthetic construct
MQYKLVICGKTLKGETTTKAVDAETAEKAFKQYANDNGVDGVWTYDDATKTFTVTE

SEQ ID NO: 4 - protein, 76 aa, synthetic construct [sequence omitted]

SEQ ID NO: 5 - protein, 62 aa, synthetic construct
MGSSHHHHHHSSGLVPRGSHGMADEEKLPPGWEKRMSRSSGRVYYFNHITNASQWERPSGNS

SEQ ID NO: 6 - protein, 123 aa, synthetic construct
MGSSHHHHHHSSGLVPRGSHMGTTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDGT

SEQ ID NO: 7 - protein, 33 aa, synthetic construct
GGGGSGSAGSEAAGSEGSAGSEAAGSEGGGGSE

SEQ ID NO: 8 - protein, 55 aa, synthetic construct
  REPEAT 6..10: note = 7 times
  REPEAT 16..20: note = 7 times
  REPEAT 16..30: note = 7 times
  REPEAT 36..40: note = 7 times
  REPEAT 46..50: note = 7 times
VPGCGVPGVGVPGTGVPGVGVPGAGVPGVGVPGTGVPGVGVPGAGVPGVGVPGTG

SEQ ID NO: 9 - protein, 55 aa, synthetic construct
  REPEAT 6..10: note = 7 times
  REPEAT 16..20: note = 7 times
  REPEAT 26..30: note = 7 times
  REPEAT 36..40: note = 7 times
  REPEAT 46..50: note = 7 times
VPGAGVPGVGVPGTGVPGVGVPGCGVPGVGVPGTGVPGVGVPGAGVPGVGVPGTG

SEQ ID NO: 10 - protein, 274 aa, synthetic construct
MGSHHHHHHSGLVPRGSSMQYKLVICGKTLKGETTTKAVDAETAEKAFKQYANDNGVDGVWTYDDATKTFTVTEGGGGSGSAGSEAAGSEGSAGSEAAGSEGGGGSENLYFQSGSHMSSTNNQLTVTNNSGVAQINAKNSGLYTTVYDTKGKTTNQIQRTLSVTKAATLGDKKFYLVGDYNTGTNYGWVKQDEVIYNTAKSPVKINQTYNVKPGVKLHTVPWGTYNQVAGTVSGKGDQTFKATKQQQIDKATYLYGTVNGKSGWISKYYLTAGS

SEQ ID NO: 11 - protein, 257 aa, synthetic construct
GSSMQYKLVICGKTLKGETTTKAVDAETAEKAFKQYANDNGVDGVWTYDDATKTFTVTEGGGGSGSAGSEAAGSEGSAGSEAAGSEGGGGSENLYFQSGSHMSSTNNQLTVTNNSGVAQINAKNSGLYTTVYDTKGKTTNQIQRTLSVTKAATLGDKKFYLVGDYNTGTNYGWVKQDEVIYNTAKSPVKINQTYNVKPGVKLHTVPWGTYNQVAGTVSGKGDQTFKATKQQQIDKATYLYGTVNGKSGWISKYYLTA

SEQ ID NO: 12 - protein, 272 aa, synthetic construct
MGSHHHHHHSGLVPRGSSMQYKLVICGKTLKGETTTKAVDAETAEKAFKQYANDNGVDGVWTYDDATKTFTVTEGGGGSGSAGSEAAGSEGSAGSEAAGSEGGGGSENLYFQSGSHMSSTNNQLTVTNNSGVAQINAKNSGLYTTVYDTKGKTTNQIQRTLSVTKAATLGDKKFYLVGDYNTGTNYGWVKQDEVIYNTAKSPVKINQTYNVKPGVKLHTVPWGTYNQVAGTVSGKGDQTFKATKQQQIDKATYLYGTVNGKSGWISKYYLTA

(19) United States
(12) Patent Application Publication
(10) Pub. No.: US 2026/0041646 A1
(43) Pub. Date: Feb. 12, 2026

(54) Nanoparticles and Method for Targeting the Nanoparticles to Bacterial Biofilms Using Bacterial Surface Proteins

(71) Applicants: Nicholas Charles Fitzkee, Starkville, MS (US); Dhanush Lakmal Yapa Amarasekara, Starkville, MS (US)

(72) Inventors: Nicholas Charles Fitzkee, Starkville, MS (US); Dhanush Lakmal Yapa Amarasekara, Starkville, MS (US)

(73) Assignee: Mississippi State University, Starkville, MS (US)

(21) Appl. No.: 19/293,975

(22) Filed: Aug. 7, 2025

Related U.S. Application Data
(60) Provisional application No. 63/681,160, filed on Aug. 9, 2024.

(51) Int. Cl. A61K 9/51; A61K 33/242; A61K 38/16; A61K 41/00; A61P 31/04

(52) U.S. Cl. CPC A61K 9/5146; A61K 33/242; A61K 38/164; A61K 41/00; A61P 31/04

(57) ABSTRACT

A thermally responsive nanosphere for binding to biofilms including a gold nanoparticle core functionalized with a polyethylene glycol; an R2ab fusion protein construct including an anchor that is a stable globular domain having a surface accessible cysteine residue, a linker, an S. epidermidis R2ab protein of SEQ ID NO. 1, and an elastin-like polypeptide. The linker may be a protein having SEQ ID NO. 7; and the anchor may be a modified third IgG binding domain from streptococcal protein G (GB3) having SEQ ID NO. 3, a ubiquitin protein having SEQ ID NO. 4, a Pin1 WW domain having SEQ ID NO. 5, and a fibronectin protein domain 3FN3 having SEQ ID NO. 6. A method for treating a biofilm containing S. epidermidis bacteria by binding a plurality of the nanospheres to the biofilm and exposing the biofilm to laser irradiation.

### STATEMENT OF GOVERNMENT INTEREST

This invention was made with government support under grant no. R01AI139479 awarded by the National Institutes of Health. The government has certain rights in the invention.

### INCORPORATION OF MATERIAL OF XML SEQUENCE LISTING BY REFERENCE

The sequence listing submitted herewith as an XML file named “MSU1018SequenceListing” created on Jul. 3, 2025, which is 17 kilobytes in size, is hereby incorporated by reference in its entirety.

### BACKGROUND OF THE INVENTION

Biofilm-related infections are associated with high mortality and morbidity, combined with significant treatment costs. Traditional antibiotics are becoming less effective against such infections due to the emergence of drug-resistant bacterial strains. The need to treat biofilms on medical implants is particularly acute, and one persistent challenge is selectively directing treatments to the biofilm site.

Biofilms are structured assemblies of bacterial cells that attach to surfaces and are enclosed in extracellular polymeric substances (EPS).¹⁻³ Compared to planktonic bacteria, bacteria in biofilms possess an altered metabolic state and physical environment.⁴⁻⁶ Staphylococcus aureus and S. epidermidis are the primary causes of implant-related biofilm infections, endocarditis, and sepsis.⁷ S. aureus produces a wide variety of virulence factors, making it more virulent than S. epidermidis.⁸ However, S. epidermidis more readily attaches to surfaces and is responsible for 40-45% of infections in hip and knee replacements.⁹ Mera et al. reported that the frequency of staphylococcal infections has risen, with hospital stays doubling from 1998-2007.¹⁰ Therefore, treatments for biofilm staphylococcal infections are still an immediate and formidable challenge, and thus new therapeutic agents to target staphylococcal biofilms is a high priority.

While traditional antibiotics are useful in controlling bacteria,¹¹ bacterial biofilms can be up to 1,000 times less sensitive to antibiotics than planktonic bacteria.¹² Drug-resistant biofilms are increasingly common.^{10,13} Nanoparticles offer great potential for solving issues of biofilm drug delivery, and new nanoparticle-based therapies are being developed to complement small-molecule antibiotics. For example, Gao et al. prepared azithromycin (AZM) functionalized clustered nanoparticles to treat biofilms. In an acidic biofilm milieu, these clustered nanoparticles can disintegrate and release a secondary AZM-conjugated nanoparticle with a smaller size and positive charge, which helps them penetrate biofilms, boosting antibiofilm activity.¹⁴ However, the EPS layer functions as a proficient physical and metabolic barrier, resulting in elevated antibiotic resistance by limiting drug penetration and triggering antibiotic inactivation. As a result, several antibiotic-free methods have been developed in recent years, including photodynamic treatment (PDT), photothermal therapy (PTT), and antibacterial therapies employing bioactive materials.¹⁵⁻¹⁸

Of these methods, photothermal therapy (PTT) has demonstrated promise in treating bacterial infections, partly because of the rapid development of photothermal agents (PTAs), the critical component of PTT. In general, PTT uses laser light to produce limited and controlled thermal damage to bacterial cells.¹⁹ PTT is efficient in treating biofilm infections and sidesteps drug resistance.²⁰ However, existing PTAs have limited ability to specifically target biofilms (e.g., on a medical implant), and the body's proteins also tend to bind nanoparticles,²¹ forming a protein corona that hinders therapeutic effectiveness.

Combining multiple functionalization strategies on a single nanoparticle shows promise for developing general-purpose PTAs. For example, Wang et al. developed biofilm-responsive caged guanidine nanoparticles (CGNs) to achieve deep biofilm penetration. CGNs use selective guanidine release, triggered by biofilm acidity, to improve biofilm attachment and penetration. Then, CGNs convert near-infrared (NIR, 800-1100 nm wavelength) laser energy into localized heat to thermally destroy bacterial biofilms. However, the synthesis of CGNs requires multiple steps, and the compounds used during the synthesis can induce cytotoxicity and neurological complications.^{22,23}

In another study, Fang et al. synthesized α-Fe₂O₃/AgAu/polydopamine (PDA) nanospindles, which exhibited antibiofilm activity by combining a controlled Ag⁺ release and a photothermal effect. These particles were 99.9% effective against E. coli and S. aureus biofilms.²⁴ However, they exhibited poor targeting of biofilms, producing undesirable inflammation in healthy tissues following NIR laser irradiation. Combining multiple functionalities improved PTA performance for both systems, yielding a synergistic benefit.

Thermally responsive nanospheres (TRNs) have been previously developed as a promising platform for treating biofilms under static conditions.²⁵ Elastin-like polypeptide (ELP) was used to functionalize citrate-capped spherical gold nanoparticles (AuNPs). This resulted in high photothermal conversion efficiencies (η˜60%) rivaling the values observed for nanorods.²⁶ E. coli and S. epidermidis biofilms were both successfully eliminated under static conditions using TRNs. By tuning the ELP transition temperature (Tₜ) the degree to which the temperature increased could be controlled upon exposure to NIR. These TRNs were also biocompatible, employing a citrate-based synthesis method. Nevertheless, these biofilms and NIR treatment were applied under static conditions, and no specific biofilm targeting was employed. A practical system will require a mechanism for targeting biofilms, which should ideally be effective under the dynamic flow conditions found in circulating blood.²⁷

### SUMMARY OF THE INVENTION

The present invention relates to a protein-based functionalization method that targets the extracellular matrix of biofilms. The protein, derived from the extracellular Staphylococcus epidermidis autolysin, directs nanoparticles to S. epidermidis cell wall components, which are not expected to be present in healthy mammalian tissues. This functionalization may be applied to a gold nanoparticle (AuNP) core, along with elastin-like polypeptides (ELPs), which generate a robust photothermal response. In addition to biofilm targeting, the particles exhibit low protein binding in the body, and the photothermal conversion can be modulated by changing the ELP transition temperature. These functionalized AuNPs strongly interact with biofilms under static and dynamic flow conditions but exhibit weak interactions with serum-coated surfaces. Near-infrared laser irradiation resulted in a 10,000-fold improvement in killing efficiency compared to untreated controls (p<0.0001). This targeting strategy provides a versatile approach to targeting drug-resistant infections and should be applicable to other anti-biofilm nanoparticle platforms.

In a first aspect, the present invention relates to a thermally responsive nanosphere for targeted binding to biofilms, wherein the nanospheres comprise:
a gold nanoparticle core functionalized with:
   a) a polyethylene glycol;
   b) an R2ab fusion protein construct including an anchor, a linker and a S. epidermidis R2ab protein having SEQ ID NO. 1,
      wherein the anchor is any stable globular domain, wherein the anchor comprises a surface accessible cysteine residue for attachment to the gold nanoparticle core, wherein the anchor is optionally selected from the group consisting of a modified third IgG binding domain from streptococcal protein G (GB3) having SEQ ID NO. 3, a ubiquitin protein having SEQ ID NO. 4, a Pin1 WW domain having SEQ ID NO. 5, and a fibronectin protein domain 3FN3 having SEQ ID NO. 6, and
      wherein the linker is optionally a protein having SEQ ID NO. 7; and
   c) an elastin-like polypeptide.

The thermally responsive nanosphere of paragraph [0011], wherein the R2ab fusion protein construct is encoded by a nucleotide sequence of SEQ ID NO. 2.

The thermally responsive nanosphere of any one of paragraphs [0011]-[0012], wherein the elastin-like polypeptide may be selected from the group consisting of SEQ ID NO. 8 and SEQ ID NO. 9.

The thermally responsive nanosphere of any one of paragraphs [0011]-[0013], wherein the polyethylene glycol is thiolated, and optionally, the polyethylene glycol has an average molecular weight of from about 1,000 g/mol to about 10,000 g/mol, or about 5,000 g/mol.

The thermally responsive nanosphere of any one of paragraphs [0011]-[0014], wherein the anchor binds to the gold nanoparticle core.

The thermally responsive nanosphere of paragraph [0015], wherein a molar ratio of the gold nanoparticle core to the elastin-like polypeptide is from about 1:25 to about 1:675, or from about 1:125 to about 1:500, or about 1:250.

The thermally responsive nanosphere of any one of paragraphs [0011]-[0016], wherein a molar ratio of the gold nanoparticle core to the polyethylene glycol to the R2ab fusion protein construct may be from about 1:25:250 to about 1:225:250, or from about 1:75:250 to about 1:225:250, or about 1:25:250, or about 1:75:250, or about 1:175:250, or about 1:225:250.

The thermally responsive nanosphere of any one of paragraphs [0011]-[0017], wherein the gold nanoparticle has a hydrodynamic diameter of from about 5 nm to 50 nm, or from about 10 nm to about 30 nm, or about 15 nm, as determined by dynamic light scattering.

The thermally responsive nanosphere of any one of paragraphs [0011]-[0018], wherein the R2ab domain may be fused to a C-terminal end of the linker.

In a second aspect, the present invention relates to a method for treating a biofilm containing S. epidermidis bacteria comprising binding a plurality of the nanospheres of any one of paragraphs [0011]-[0019] to the biofilm and exposing the biofilm with the bound nanoparticles to laser irradiation.

1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows in the top portion a schematic illustration of the stepwise synthesis of a thermally-responsive nanosphere (TRN) assembly AuNP@PEG5K@R2abF@ELPA4C, of the present invention. The bottom portion of FIG. 1 shows the application of the final nano assembly of AuNP@PEG5K@R2abF@ELPA4C for targeting and killing an S. epidermis biofilm using 808 nm near-infrared (NIR) laser light. ELPA4C refers to an elastin-like peptide into which cysteine residues were introduced at position 4 of the first repeat (A4C).

FIGS. 2A-2H show the physiochemical characterizations of AuNP@PEG5K@R2abF@ELPA4C when prepared at different molar ratios. The data points are shown, and the error bars represent the standard error of the mean (SEM) for at least three independently prepared samples. Statistical significance between grouped samples was determined using one-way ANOVA and Tukey's multiple comparisons test (****, p<0.0001)

FIG. 2A shows Dynamic Light Scattering (DLS) profiles of AuNP@PEG5K@R2abF to optimize conditions for PEG5K concentration. D_{H} denotes hydrodynamic diameter (nm).

FIG. 2B shows a chart with the polydispersity index of the various AuNP@PEG5K@R2abF samples.

FIG. 2C shows the affinity profiles of AuNP@PEG5K@R2abF on polystyrene surfaces as measured by a plate reader using λ=530 nm.

FIG. 2D shows DLS measurements on AuNP@PEG5K@R2abF, at 20 nM or 240 ppm Au, for 5 cycles each. A reversible, temperature dependent agglomeration was observed.

FIG. 2E shows the effects of different elastin-like polypeptide (ELPA4C) concentrations on affinity profiles of AuNP@PEG5K@R2abF@ELPA4C on polystyrene surfaces measured by a plate reader using λ=530 nm.

FIG. 2F shows the hydrodynamic diameter (D_{H}, nm) of various AuNPs.

FIG. 2G shows a UV-vis extinction plots for various AuNPs.

FIG. 2H shows zeta potentials (mV) of functionalized AuNPs, AuNP@PEG5K, AuNP@PEG5K@R2abF, and AuNP@PEG5K@R2abF@ELPA4C.

FIGS. 3A-3C show the colloidal stability and cytotoxicity analyses of nano-assemblies. The error bars represent the SEM of three experiments, and all data points are shown.

FIG. 3A shows the observed hydrodynamic diameter (D_{H}) of the nano-assemblies AuNP, AuNP@PEG5K, AuNP@GB3NF, AuNP@ELPA4C, AuNP@PEG5K@R2abF, and AuNP@PEG5K@R2abF@ELPA4C, with and without 30% (v/v) fetal bovine serum. In this example, significance was assessed using one-way ANOVA with Tukey's multiple comparisons test (ns, not significant; ***, p<0.001; ****, p<0.0001).

FIG. 3B shows the growth curves of S. epidermidis bacteria in the presence of 20 nM (120 ppm of Au) AuNPs.

FIG. 3C shows the effect of different concentrations of nano-assemblies on S. epidermidis cell growth measured by counting the colony forming units (CFU mL⁻¹).

FIGS. 4A-4B show the results of determination of the adherence of targeted thermally responsive nanospheres (TRNs) on different biological surfaces.

FIG. 4A shows affinity profiles of nano-assemblies on biofilms and serum-coated polystyrene surfaces as determined by inductively coupled plasma-mass spectrometry (ICP-MS). Significance in panel A was assessed using one-way ANOVA with Tukey's multiple comparisons test (ns, no significant; *, p<0.05; **, p<0.01). The error bars represent the SEM of three experiments and all data points are shown.

FIG. 4B shows a Scanning Electron Microscopy (SEM) image of an S. epidermidis biofilm formed on a polystyrene surface treated with AuNP@PEG5K@R2abF@ELPA4C and energy dispersive X-ray spectroscopy (EDS) elemental analysis images. EDS indicates the presence of carbon (C), oxygen (O), and gold (Au) on the surface of the treated biofilms.

FIGS. 5A-5E show the results of testing the anti-bacterial ability of AuNP@PEG5K@R2abF@ELPA4C.

FIG. 5A shows temperature evolution curves of 10 nM (120 ppm Au) AuNP@PEG5K, AuNP@PEG5K@R2abF, and AuNP@PEG5K@R2abF@ELPA4C added to S. epidermidis biofilms under NIR Irradiation above the transition temperature (Tₜ).

FIG. 5B shows temperature evolution curves of 20 nM (240 ppm Au) AuNP@PEG5K, AuNP@PEG5K@R2abF, and AuNP@PEG5K@R2abF@ELPA4C added to S. epidermidis biofilms under NIR Irradiation above the transition temperature (Tₜ).

FIG. 5C shows temperature evolution curves of 30 nM (360 ppm Au) AuNP@PEG5K, AuNP@PEG5K@R2abF, and AuNP@PEG5K@R2abF@ELPA4C added to S. epidermidis biofilms under NIR Irradiation above the transition temperature (Tₜ).

FIG. 5D shows the results of a serial dilution assay on agar plates demonstrating how colony-forming units (CFU) mL⁻¹ was determined.

FIG. 5E shows the CFU mL⁻¹ of S. epidermidis present in biofilms after treatment with targeted TRNs at 10-30 nM particle concentrations, where AuNP@PEG5K and AuNP@PEG5K@R2abF were used as controls. Significance was assessed using one-way ANOVA with Tukey's multiple comparisons test (n.s., not significant; ****, p<0.0001). The error bars represent the SEM of three experiments, and all data points are shown.

FIGS. 6A-6J show the results of testing the anti-bacterial ability of AuNP@PEG5K@R2abF@ELPA4C in a dynamic flow system.

FIG. 6A shows a schematic diagram of a dynamic flow system used to test the adherence of AuNP@PEG5K@R2abF@ELPA4C under flow.

FIG. 6B shows an actual image of the dynamic flow system used in FIG. 6A.

FIG. 6C shows visible biofilm formation on plastic tubes (3 cm) after flowing BHI media containing S. epidermidis cells in contact with the plastic tubes for 48 hours.

FIG. 6D shows crystal violet staining used to determine the presence of S. epidermidis biofilm on the inner walls of the plastic tubes.

FIG. 6E shows the presence of AuNP@PEG5K@R2abF after flowing 20 nM AuNP@PEG5K@R2abF through the biofilm containing plastic tubes for 12 hours.

FIG. 6F shows the presence of AuNP@PEG5K@R2abF@ELPA4C after flowing 20 nM AuNP@PEG5K@R2abF@ELPA4C through the biofilm containing plastic tubes for 12 hours.

FIG. 6G shows an image of a 3D printed structure used to measure the temperature evaluation curves of the plastic tubes under the NIR irradiation.

FIG. 6H shows a thermal camera image of the 3D printed structure of FIG. 6G.

FIG. 6I shows temperature evaluation curves of the plastic tubes after irradiation with 1.8 W/cm² NIR laser for 15 minutes.

FIG. 6J shows colony formation after the treatment with the NIR laser for S. epidermidis, S. epidermidis & AuNP@PEG5K, and S. epidermidis & AuNP@PEG5K@R2abF@ELPA4C.

FIG. 7 shows the particle size distribution (PSD) of 15 nm citrate AuNPs, as determined by an Anton Paar Dynamic Light Scattering (DLS) system at room temperature. The average hydrodynamic diameter (D_{H}) and polydispersity index (PDI %) are also shown.

FIG. 8 shows a transmission electron microscopy (TEM) image of 15 nm synthesized citrate AuNPs having a uniform size distribution, as determined by a JEOL 2100 TEM instrument.

FIG. 9 shows an extinction spectrum of 15 nm AuNPs as a function of wavelength, which was measured using an Olis-refurbished, Peltier-controlled Agilent 8453 UV-vis spectrophotometer. The single peak at 520 nm indicates uniform, spherical AuNPs with a diameter of approximately 15 nm.

FIG. 10 shows an amino acid sequence of R2ab fusion protein (R2abF) (SEQ ID NO: 11), wherein the Cysteine residue is highlighted and printed in red text (see first C, 11 amino acids in on the top line).

FIG. 11 shows ¹⁵N-¹H 2D TROSY spectra collected for the protein constructs of the present invention. An overlay of the spectra for R2abF is shown in yellow, R2ab is shown in red, and GB3NF is shown in blue. No significant shifts were observed for peaks in the R2ab and GB3 domains, indicating that there was no structural perturbation to the domains when connecting them via a flexible linker.

FIG. 12 shows a transmission electron micrograph (TEM) image of targeted thermally-responsive nanospheres (AuNP@PEG5K@R2abF@ELPA4C) which demonstrated that the TRNs are not aggregated below the transition temperature (Tₜ).

FIG. 13A shows an SEM image of S. epidermidis biofilm on polystyrene without the targeted TRNs.

FIG. 13B shows an EDS image of an S. epidermidis biofilm on polystyrene without targeted TRNs. The elemental map from the EDS highlighted the absence of a significant amount of gold (Au). It was noted that Pt appears since it is used in the sputter coating process for preparing SEM samples.

FIG. 14A shows an SEM image of S. epidermidis biofilm on polystyrene after treatment with targeted TRNs. The biofilms were treated with AuNP@PEG5K@R2abF@ELPA4C then washed before preparation to identify whether targeted TRNs tightly bind to biofilms.

FIG. 14B shows EDS image of S. epidermidis biofilm on polystyrene after treatment with the targeted TRNs. The elemental map from the EDS image highlights the presence of gold (Au) peaks.

FIG. 15 shows a UV-vis extinction spectra of AuNP@PEG5K@R2abF@ELPA4C before and after five cycles of heating above Tₜ and cooling below the Tt. The spectra retain its shape after repeated cycles of aggregation.

FIG. 16 shows the photothermal effect of AuNP@PEG5K@R2abF@ELPA4C when exposed to laser irradiation. The chart shows the temperature change with laser irradiation (on) and without (off) when 20 nM (240 ppm Au) aqueous solution was irradiated with 1.8 W cm⁻², 808 nm laser at 39° C. The exponential decay fitting to determine the photothermal conversion efficiency (η) is shown in red.

FIG. 17 shows the aggregation of AuNP@PEG5K@R2abF@ELPA4C when prepared above Tₜ, as determined by TEM. The aggregation leads to enhanced photothermal conversion efficiency of photons in the near-infrared (NIR).

FIG. 18 shows an SEM image of S. epidermidis biofilms after 72 hours of continuous dynamic flow through polystyrene tubes.

FIG. 19 shows the affinity of differently functionalized AuNPs with a polystyrene surface. Each well was stained with AuNP@PEG5K and AuNP@PEG5K@R2abF, where different ratios of R2abF and PEG5K were used on each AuNP. The first column shows no staining because AuNP@R2abF rapidly aggregates without prior functionalization with PEG5K and the aggregate washes out of the well. The final column shows that AuNPs lacking R2ab do not stain polystyrene.

FIG. 20 shows the selective adhesion of the targeted TRNs to S. epidermidis biofilms with varying ratios of the elastin-like polypeptide (ELP). After washing, the persistence of the targeted TRNs was determined in a plate read by monitoring the intensity of the pink color (λ=530 nm). The AuNPs coated with only PEG (green) exhibited the worst adhesion properties to the biofilm.

FIG. 21 shows SEM and EDS images of an S. epidermidis biofilm on polystyrene with targeted TRNs. The images shown in FIG. 21 are of S. epidermidis biofilms after treatment with AuNP@PEG5K@R2abF@ELPA4C. Elemental analysis indicated the presence of nitrogen (N, from the biological sample), and platinum (Pt, from putter coating during sample preparation) on the surface of the biofilm.

FIGS. 22A-22C show structural features of the S. epidermidis biofilm proteins used in the present invention.

FIG. 22A shows the topology of accumulation associated protein (Aap) and autolysin E (AtlE). Red arrows indicate sites of posttranslational cleavage. In Aap, the B-repeats and the Pro/Gly-rich (PGR) regions are highlighted, as are the amidase domains in AtlE.

FIG. 22B shows the structure of a single Aap B-repeat as well as a diagram of the Brpt1.5 construct used in the examples.

FIG. 22C shows crystal structures of the AM catalytic domain and R1 domain from AtlE, as well as the Aap Brpt 1.5 construct.

FIG. 23 shows a vector comprising a nucleotide sequence (SEQ ID NO: 2) encoding a GB3-R2ab Fusion protein construct (SEQ ID NO: 10) of the present invention.

FIG. 24 shows the DNA sequence (SEQ ID NO: 2) of a GB3-R2ab Fusion construct of the present invention.

FIG. 25 shows a protein sequence (SEQ ID NO: 12) of the GB3-R2ab Fusion construct of the present invention.

FIG. 26 shows the functionalization method of the present invention for targeting photothermal nanoparticles to biofilms.

### DETAILED DESCRIPTION OF THE INVENTION

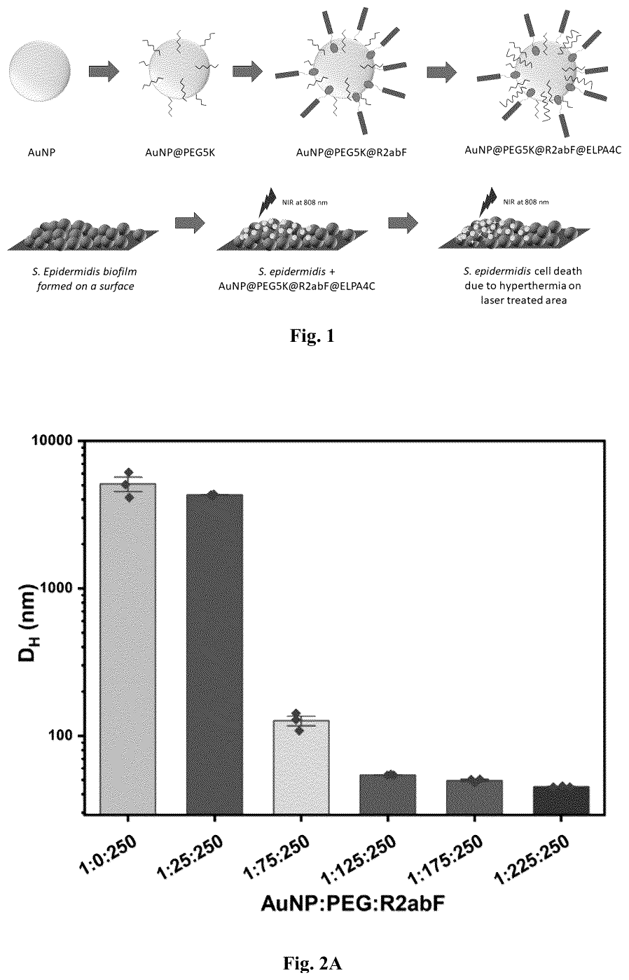

The present invention relates to biofilm-targeting TRNs designed to treat S. epidermidis infections (FIG. 1), extending the application range of TRNs. The TRNs employ a protein-based localization strategy whereby the S. epidermidis R2ab domain drives TRN binding to S. epidermidis biofilms. R2ab is a subdomain of the autolysin enzyme (AtlE), and it binds to the cell wall, particularly to the lipoteichoic acid (LTA) and wall teichoic acid (WTA) at the septum of dividing cells.^{28,29} R2ab can favorably interact with LTA and peptidoglycan (PGN) in the staphylococcal cell wall, and its normal function supports bacterial cleavage.²⁸ The present method exploits this property so that TRNs can specifically target the cell wall of S. epidermidis biofilms. Mammalian cells lack a cell wall which makes this method particularly effective. In addition, the TRNs of the present invention show low protein binding and high colloidal stability in the presence of serum. These targeted TRNs were tested under various in vitro conditions, including those where the nanoparticles are suspended in a flowing solution. It was determined that the anti-biofilm properties of the TRNs are retained even when the TRNs are flowing past established biofilms in a tubular flow cell reactor. Moreover, binding to serum-coated surfaces is relatively low, demonstrating that R2ab-functionalized TRNs have targeting specificity. The biofilm targeting TRNs disclosed herein exhibit outstanding therapeutic efficacy with reduced drug resistance in treating staphylococcus biofilm-associated infections.

### Preparation of 15 nm Gold Nanoparticles (AuNPs)

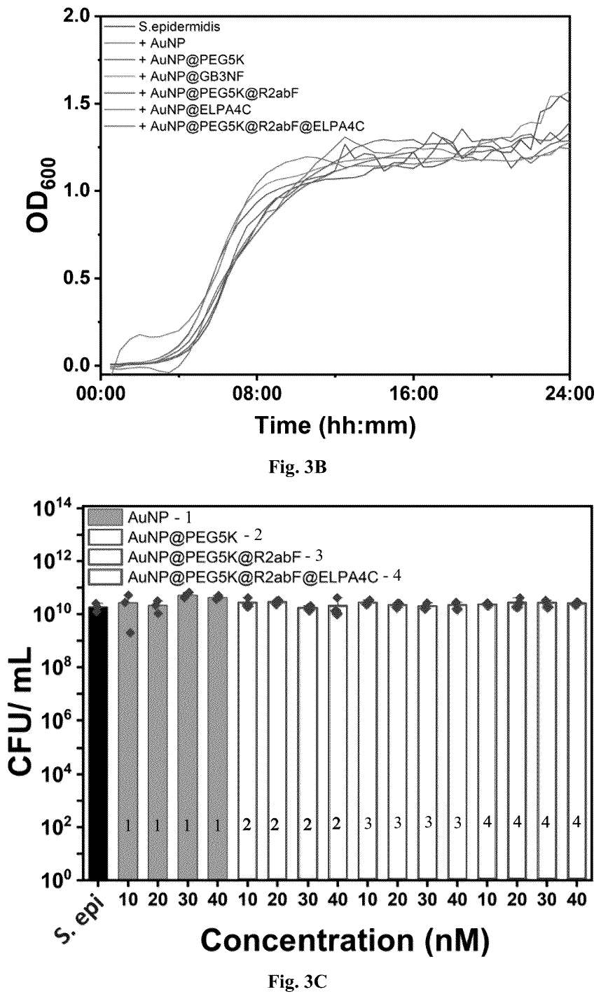

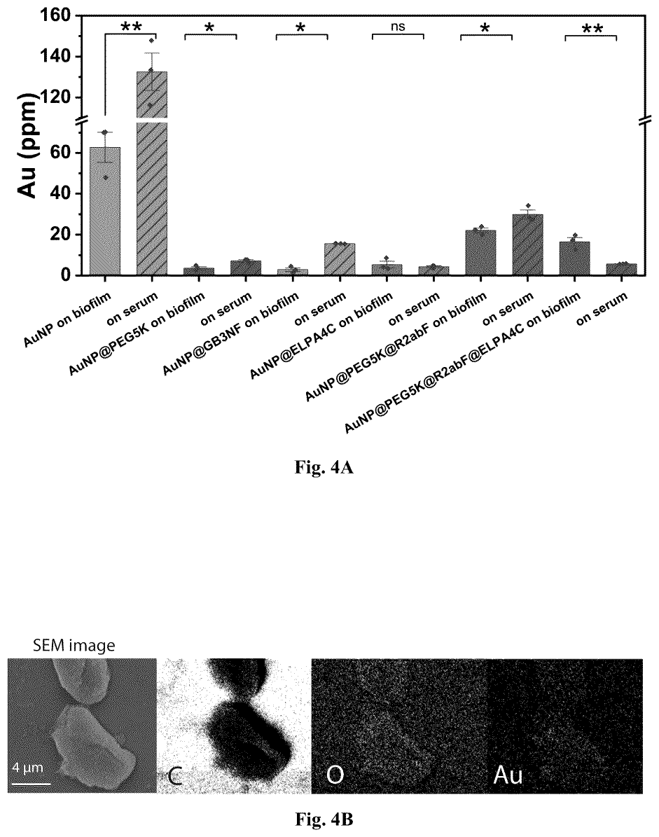

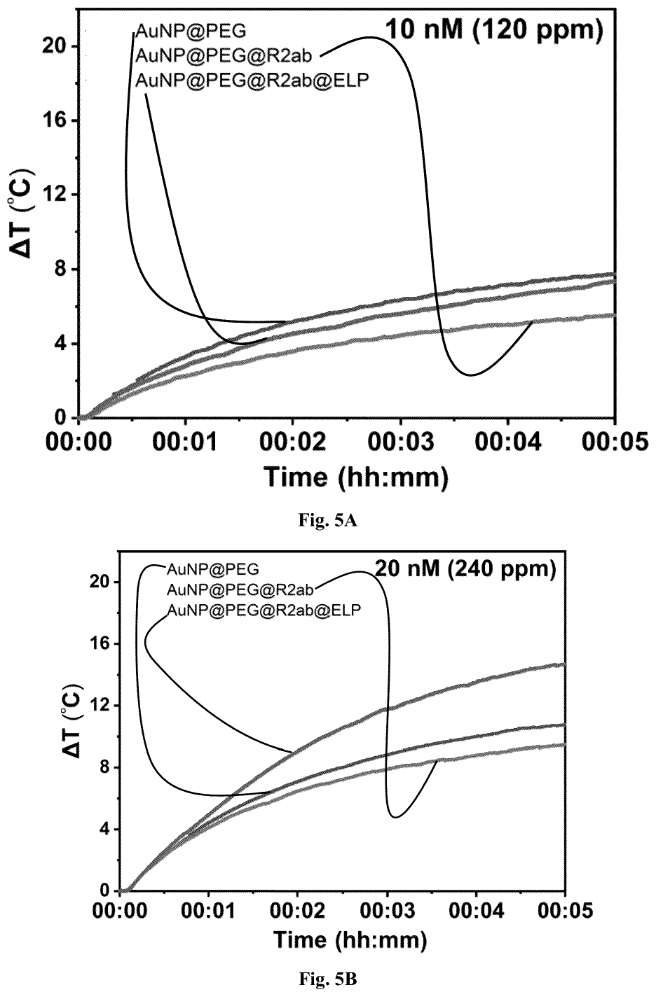

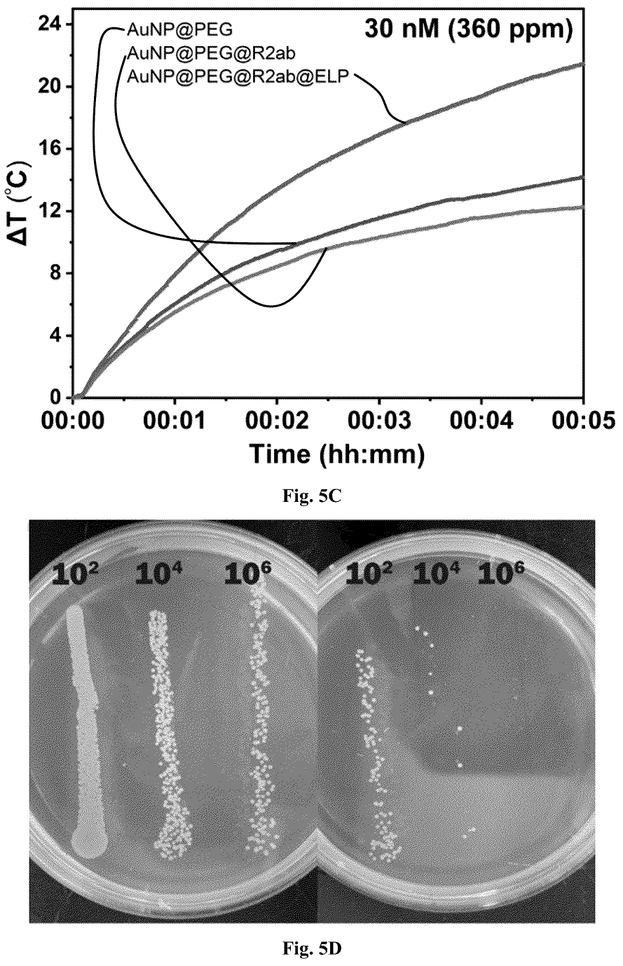

Gold nanoparticles were synthesized as described previously.^{25,30-32} The concentrated samples were characterized for size and conformity.³³ The size of the synthesized AuNPs was measured using dynamic light scattering (DLS; Anton Paar Litesizer 500) and transmission electron microscopy (TEM; JEOL 2100), and they consistently measured 15 nm in diameter (FIGS. 7 and 8). The UV-vis spectrum of synthesized AuNPs showed an intense band at 520 nm (FIG. 9), which was used to estimate the concentration of the AuNPs.^{25,33} Inductively coupled plasma-mass spectrometry (ICP-MS, PerkinElmer ELAN DRC II) was used to determine the Au concentration.^{34,35} The concentrated AuNPs were stored at 4° C. until used. In some embodiments, the concentration of the Au is referenced as particle concentration (nM) or total Au concentration (ppm).

### Preparation of the Elastin-Like Polypeptide (ELP) Construct

The 40-repeat ELP sequence consists of 40 VPGXG repeats, where X is V for all but six of the repeats. For conjugation to AuNPs, cysteine residues were introduced at position 4 of the first repeat (A4C). The protein was expressed and purified according to previously published methods.^{36,38} The complete protein sequence is available in Amarasekara et al.²⁵ The protein was stored in 20 mM 4-(2-hydroxyethyl)-1-piperazinecthanesulfonic acid (HEPES), 5 mM NaCl, and 5 mM TCEP at −20° C. for short periods (<1 month) and lyophilized for longer periods after dialyzing in deionized water. For example, the ELPs may be selected from SEQ ID No. 8 and 9.

### Preparation of the Anchor Protein GB3 Null-Fusion (GB3NF) Protein Construct

The GB3 null-fusion construct (GB3NF) is a small globular protein consisting of the third IgG-binding domain of streptococcal protein G.³⁹ It consists of 56 amino acid residues. A cysteine residue was introduced in the first β-turn (N8C). The protein was expressed and purified as described previously.²⁵ It was stored in 20 mM HEPES, 5 mM NaCl, and 5 mM TCEP.

In some embodiments, the anchor protein may be any stable globular domain, wherein the anchor comprises a surface accessible cysteine residue for attachment to the gold nanoparticle core, wherein the anchor is optionally selected from a modified third IgG binding domain from streptococcal protein G (GB3), Ubiquitin, Pin1 WW domain, and Fibronectin domain 3FN3. Ubiquitin is a small, 76 residue human protein that is involved in protein turnover. Pin1 WW domain is a small, ˜40 residue human protein domain that is involved in recognizing peptides. It is also another protein fragment, and it's naturally tethered to a larger prolyl isomerase domain. This tether lends itself to being tethered to S. epidermidis R2ab. The Pin1 WW domain is also suitable for binding to gold nanoparticles. Fibronectin domain 3FN3 is a third type-3 domain of human fibronectin which is involved in clotting and thus, found in blood plasma. Since fibronectin is already present in human blood, coating a nanoparticle, such as a gold nanoparticle, with fibronectin results in a particle that resembles the body's own protein, thus reducing possible adverse interactions.

### Preparation of the R2Ab Fusion (R2abF) Construct

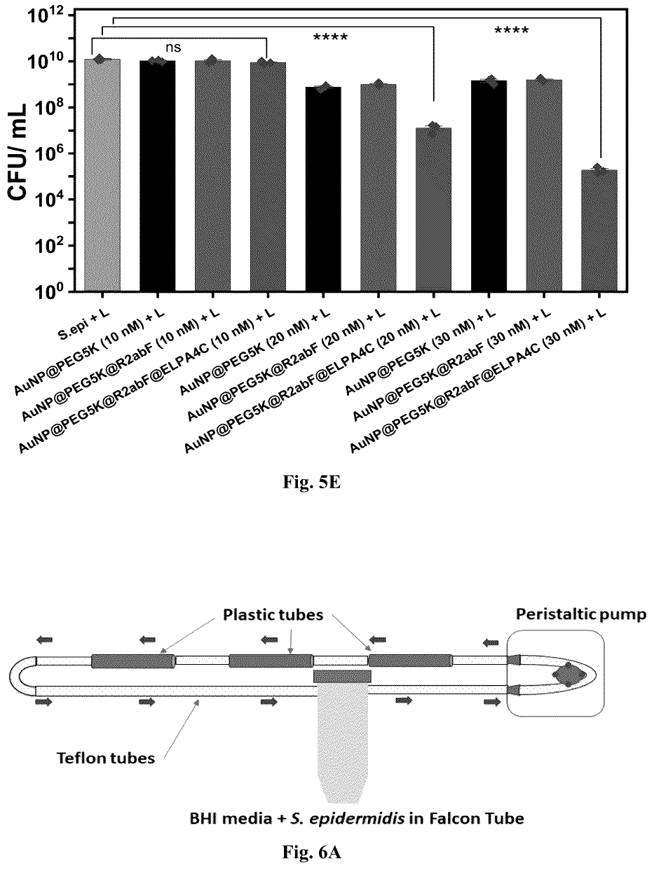

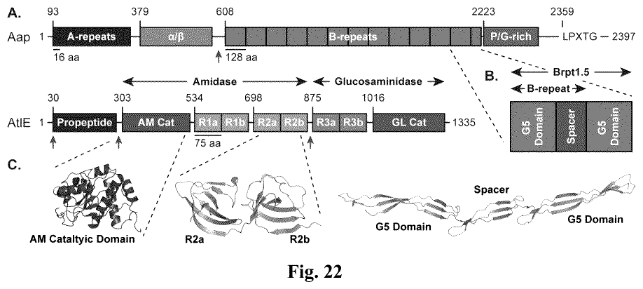

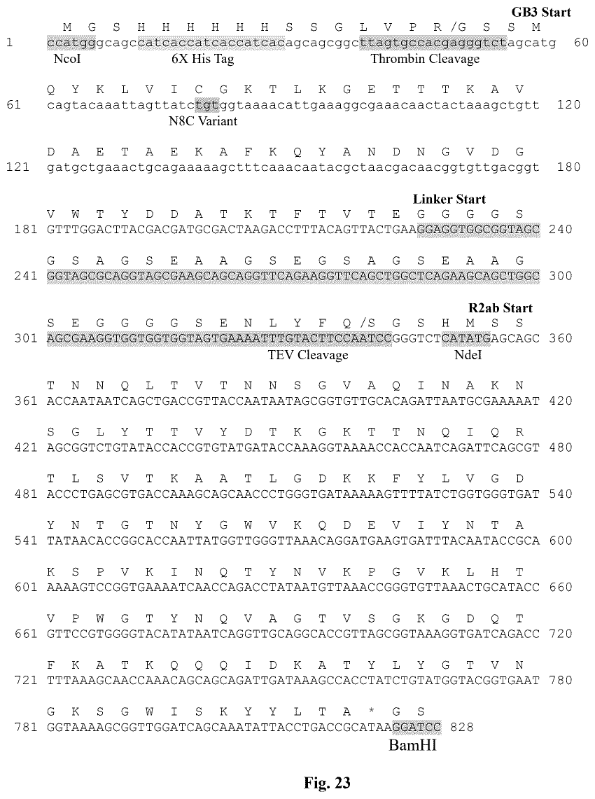

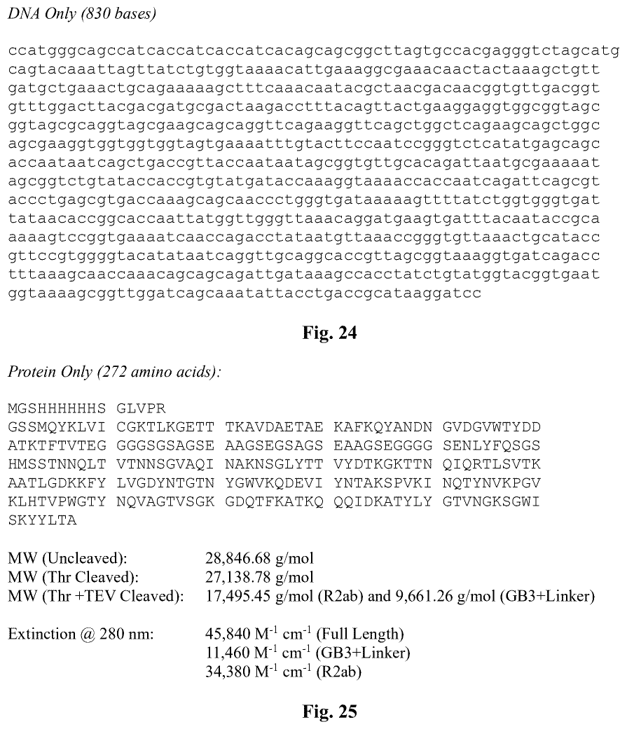

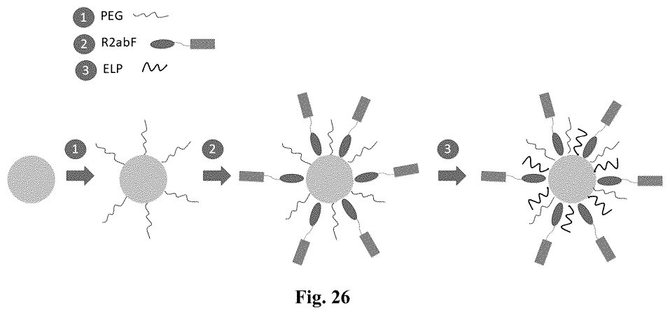

The R2ab fusion protein (R2abF) construct consists of three regions: an anchor protein (for example, a GB3 domain), a linker, and an R2ab domain (FIG. 23). The GB3 domain is similar to GB3NF, except that it contains an N8C variant to facilitate AuNP attachment. The N-terminus of the GB3 domain contains a 6× histidine tag and a thrombin cleavage site to aid purification using affinity chromatography. The linker region is designed to separate the GB3 and R2ab domains, providing a flexible linker and minimizing interactions between R2ab and the (GB3-coated) AuNP surface. For example, suitable linkers may include glycine, serine, alanine, and aspartic or glutamic acid residues, and combinations thereof. The linker may include at least 15 residues. The presence of glycine and alanine residues may be particularly useful in ensuring flexibility, while the presence of aspartic or glutamic acid residues may assist in solubility. The R2ab domain was fused at the C-terminal end of the linker. The fusion protein was subcloned into a pET-15b vector for recombinant protein expression in Escherichia coli under the control of the T7 promoter. Plasmid construction was performed by GenScript (Piscataway, NJ). Purification of R2abF was performed using the approach described previously for the isolated R2ab domain.⁴⁰ After purification, R2abF was dialyzed into a buffer containing 20 mM HEPES, 5 mM NaCl, and 5 mM TCEP. The structure of the R2ab and GB3 domains was confirmed by comparing the ¹H-¹⁵N HSQC spectrum of R2abF with the spectra of the isolated domains (FIG. 11). Chemical shift perturbations were minor in the fusion protein.

### Synthesis of Biofilm-Targeting TRNs (AuNP@PEG5K@R2abF@ELPA4C)

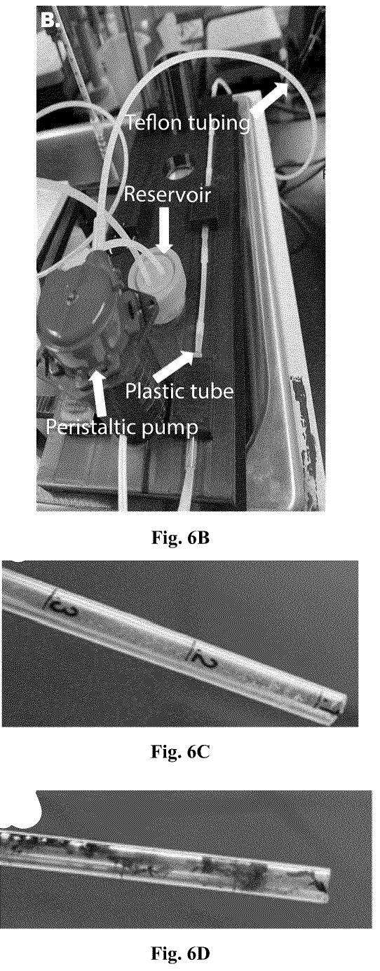

Polyethylene glycol (PEG) was thiolated to form a thiolated PEG having an average molecular weight of 5,000 g/mol and was used to coat AuNPs as described in the art.²⁵ AuNPs (AuNP@PEG5K) with different AuNP/PEG5K ratios were mixed with R2abF protein to find the optimum conditions to prevent aggregation caused by direct interaction of R2abF with the AuNP surface. The R2abF concentration was kept constant (AuNP/R2abF=1:250), and shortly after mixing R2abF with the AuNP@PEG5K, the mixture was vortexed several times to prepare the AuNP@PEG5K@R2abF in 20 mM HEPES at pH 6.5 and 5 mM NaCl. Finally, the desired concentration of ELP was added to the mixture to prepare AuNP@PEG5K@R2abF@ELPA4C. After incubation for 2 hours, the solutions were centrifuged three times at 21,300 g for 24 minutes to remove unbound PEG5K, R2abF, and ELPA4C, which remained in the supernatant. The pH of the solutions was confirmed by pH-indicator strips (EMD Millipore) using the supernatant. After the third wash, the pellet was redispersed in 20 mM HEPES pH 6.5 and 100 mM NaCl to provide a suitable environment for phase separation. AuNPs were modified with thiolated PEG5K, GB3NF, and ELPA4C separately to form AuNP@PEG5K, AuNP@GB3NF, and AuNP@ELPA4C as controls. The final concentration of Au was determined using inductively coupled plasma-mass spectrometry (ICP-MS), as described above. DLS was used to assess the nanoparticle hydrodynamic diameter (D_{H}), polydispersity index (PDI), and zeta potential. The behavior of prepared AuNP@PEG5K@R2abF@ELPA4C nano-assemblies was characterized using TEM (FIG. 12).

Interactions of Biofilm-Targeting TRNs with S. epidermidis Biofilms

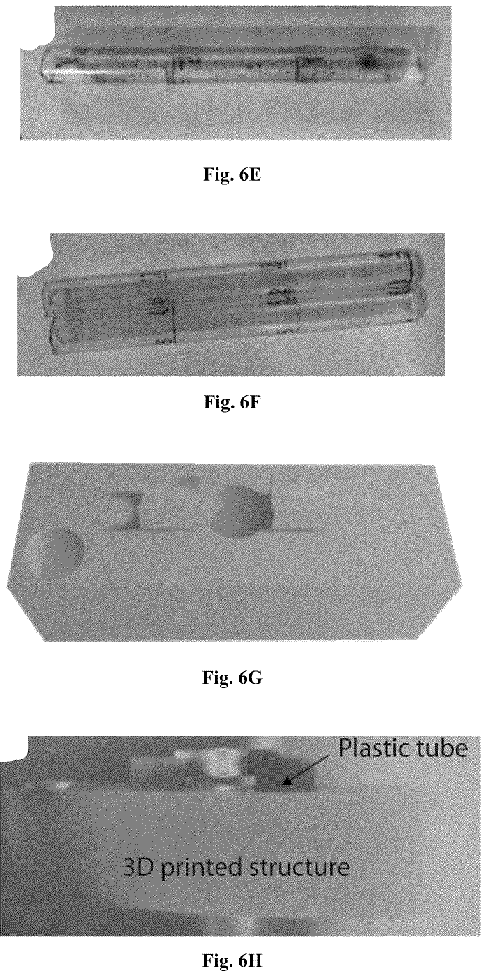

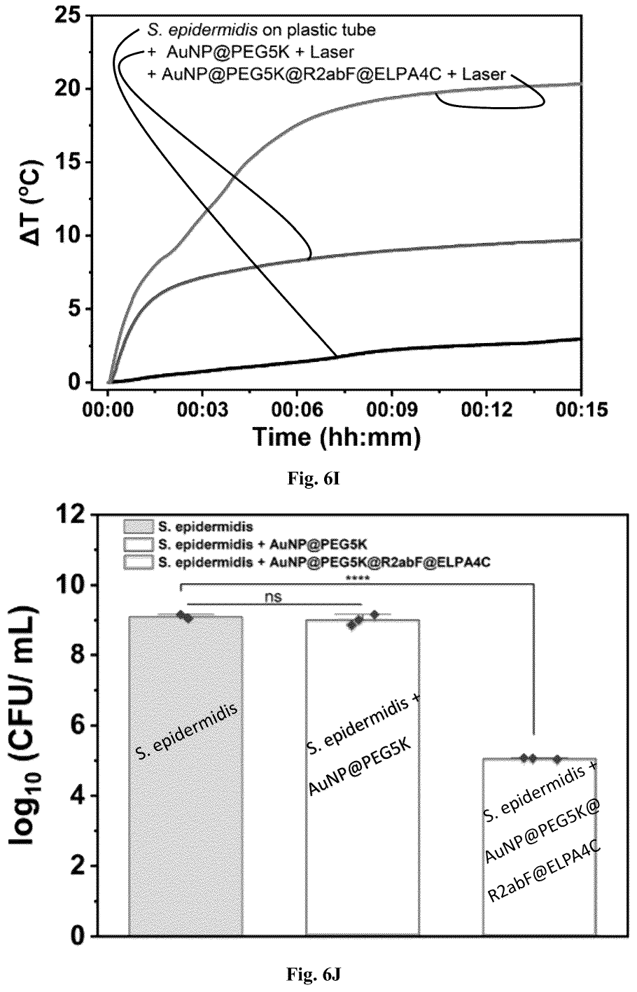

S. epidermidis cells were incubated in a brain heart infusion (BHI) medium at 37° C. and allowed to grow for 48 hours on a 96-well plate. Following the incubation, the excess planktonic cells were removed by gently washing the plate three times with phosphate-buffered saline (PBS) buffer. Then AuNP@PEG5K@R2abF@ELPA4C (100 μL) at various concentrations was added to each well and incubated for 12 hours at 37° C. Subsequently, each well was washed three times with PBS, and the presence of AuNP@PEG5K@R2abF@ELPA4C was measured using a Cytation5 plate reader (Biotec) at 25° C. by monitoring the extinction at 520 nm. The morphology of bacterial cells in the presence and absence of AuNP@PEG5K@R2abF@ELPA4C was observed using scanning electron microscopy (SEM) and energy-dispersive X-ray spectroscopy (EDS; FIGS. 13 and 14).²⁵ ICP-MS was used to measure the total gold content in each sample well. Each well was treated for 12 hours with aqua regia and then diluted to 10 mL with ultrapure water. Standard curves were generated using several dilutions of a commercial gold standard (Fluka #38168). Samples were analyzed using a PerkinElmer ELAN DRC II ICP-MS system with an autosampler. The total gold concentration in each sample (ppm or μg mL⁻¹) was calculated by interpolating the standard curve.

### Thermoresponsive Behavior of Bacterial Targeting TRNs

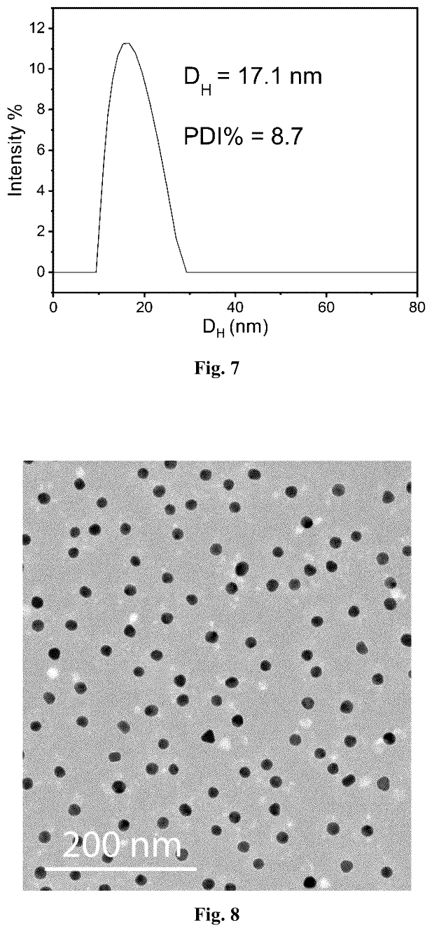

The aggregation of the AuNP@PEG5K@R2abF@ELPA4C at various temperatures was examined using DLS.²⁵ Measurements of agglomeration reversibility were performed below and above the transition temperature (Tₜ) of the AuNP@PEG5K@R2abF@ELPA4C system after equilibrating for 15 minutes for 5 cycles. UV-vis measurements were obtained before and after 5 cycles to see structural changes to the AuNP@PEG5K@R2abF@ELPA4C variant that occurred during heating and cooling cycles (FIG. 15).

### Transmission Electron Microscopy (TEM)

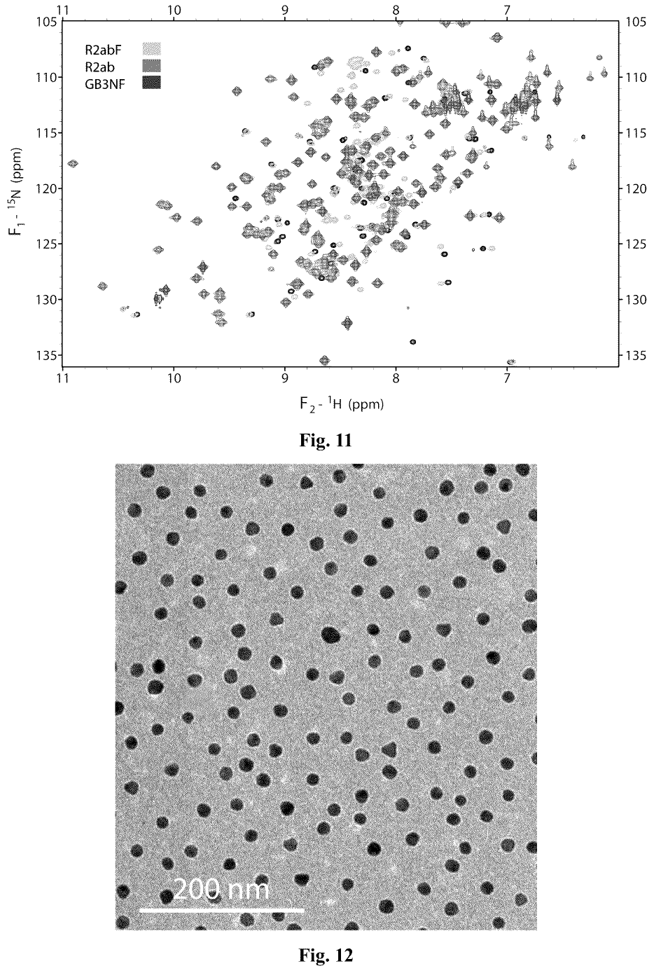

On Formvar-coated copper grids, 5 μL aliquots of a 2 nM (24 ppm of Au) AuNP@PEG5K@R2abF@ELPA4C solution were deposited. The grids were kept at the Tₜ and then exposed to NIR irradiation at 808 nm using a laser at 1.8 Wcm⁻² until the excess liquid evaporated. Prepared grids were imaged using a JEOL 2100 with an accelerating voltage of 200 kV (FIG. 17). TEM was performed at the Institute for Imaging and Analytical Technologies (I2AT) at Mississippi State University.

### Photothermal Effect of Bacterial Targeting Nano-Assemblies

AuNP@PEG5K@R2abF@ELPA4C samples (400 μL) at different gold concentrations (120-360 ppm of Au) in disposable cuvettes were incubated at their Tₜ for 15 minutes and then exposed to irradiation at 808 nm with a laser at 1.8 W cm⁻². This laser power was selected based on the temperature changes observed in previous work.²⁵ The sample temperatures were recorded using an infrared thermographic camera every 100 ms (Optris PI400i). To calculate the photothermal conversion efficiency (η) of AuNP@PEG5K@R2abF@ELPA4C, 1 mL of a 20 nM (240 ppm of Au) sample was incubated at its Tₜ for 15 minutes and then exposed to laser irradiation (808 nm, 1.8 W cm⁻²). When the temperature reached the maximum, the laser was switched off. The parameter η was calculated as previously reported.^{25,41-43}

### In Vitro Antibiofilm Activity of Bacterial Targeting Nano-Assemblies Irradiated by NIR Light.

S. epidermidis was cultured in BHI media at 37° C. and allowed to grow overnight with shaking. A seed culture was prepared from the overnight culture with an OD₆₀₀ of 0.2, and 100 μL was added to 96-well plates. Biofilm formation was accomplished by incubating the plate statically at 37° C. for 48 hours. Following the incubation period, extra cells were removed by washing three times with 100 μL of PBS buffer. Following the creation of the biofilm, the wells were treated with 100 μL of nanoparticle or control solutions, maintained at 37° C. for 15 minutes, and then subjected to 808 nm laser irradiation at 1.8 W cm⁻² for 5 minutes. After NIR irradiation, the number of colony-forming units was determined as described below.

### Dynamic Biofilm Formation on Plastic Tubes Using a 3D Printed Flow System.

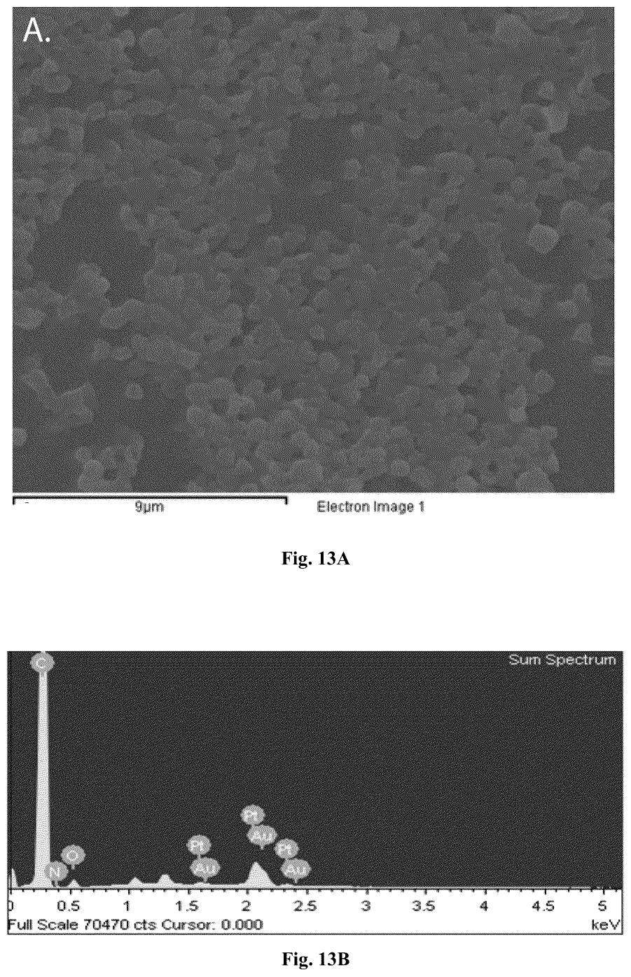

A Crealty3D CR-10 printer (Shenzhen Creality 3D Technology Co., Ltd., Shenzhen, China) was used to 3D print the flow cell reactor. The design of these devices was created using Autodesk Fusion360 3D CAD Software (Online version). Design files for the flow cell are available as part of the datasets stored online (see Zenodo link below). Before use, all components of the flow reactor were sterilized by immersion in ultrapure water and autoclaving for 15 minutes using the wet cycle. The system was built to accommodate three sterile, 3 cm untreated polystyrene tubes in series (cut from a KIMBLE serological pipette). BHI medium was inoculated with S. epidermidis strain 1301.⁴⁴ A 12 V peristaltic pump (ALAMSCN #AL12537) circulated inoculated medium through the system for 48 hours at 37° C., establishing a biofilm on the plastic tubes. After each experiment, the system was washed with sterile PBS for 5 minutes to remove any excess medium. Visible biofilm formation was observed on the inner walls of the plastic tubes. Biofilm formation on the inner walls of the plastic tubes was further confirmed using crystal violet staining and SEM imaging (FIG. 18). Next, 20 mL of AuNP, AuNP@PEG5K, and AuNP@PEG5K@R2abF@ELPA4C (30 nM or 360 ppm of Au) were circulated through the flow cell reactor separately for 12 hours to determine the affinity of these nano-assemblies on S. epidermidis biofilm under dynamic flow conditions. The tubes treated with AuNP@PEG5K and AuNP@PEG5K@R2abF@ELPA4C were then exposed to 808 nm laser irradiation at 1.8 W cm⁻² for 15 minutes.

To estimate cell viability after treatment, each plastic tube was immersed in 1 mL of PBS medium in a well of a 24-well plate for 30 minutes with shaking, allowing the live cells to shed into the PBS. Next, the tubes were removed from each well, and the number of colony-forming units was determined using a standard dropwise assay, as described below.

Determination of Colony-Forming Units Per mL (CFU mL⁻¹) in Static Biofilms

A standard dropwise assay was used to determine bacterial cell viability after treatment of static biofilms. Cells were grown in sterile 96-well plates in BHI media, as described above. After NIR irradiation, the nanoparticle solution was removed from each well of a 96-well plate, and 100 μL of sterile PBS was added to each well. Then, biofilms were carefully resuspended in each well by mixing with a pipette. The suspensions were serially tenfold diluted with sterile PBS, and 10 μL diluted samples were spread on agar plates. After incubation at 37° C. for 18 hours, the formed colonies were counted. The first serial dilution where 10-50 isolated colonies were present was used to estimate the CFU mL⁻¹ of the initial resuspended biofilm sample.

Unless otherwise stated, all measurements were reported as average and the standard error of the mean for at least three independently prepared samples. When technical replicates were used instead, this was explicitly noted. Unless otherwise noted, comparisons were performed using one-way ANOVA with GraphPad Prism 10 software. Pairwise post-hoc testing was performed using Tukey's multiple comparisons test, and statistical significance was reported at the α=0.05 significance level.

### Synthesis and Characterization of Biofilm-Targeted TRNs

The stepwise synthesis of the bacteria-targeting, thermally responsive nanosphere (TRN) of the present invention is shown in FIG. 1. For the synthesis, 15 nm diameter spherical citrate-AuNPs were used (FIGS. 7 and 8). R2ab was chosen to functionalize the AuNPs (AuNP@R2ab), enabling AuNPs to bind the cell wall components of S. epidermidis. However, a major challenge with these functionalizing techniques is the potential of losing protein activity upon interaction with the surface of the nanoparticle.⁴⁵⁻⁴⁷ For this reason, optimization of the orientation and accessibility of the active site of R2ab to its binding surface in solution were determined when the protein was conjugated to the nanoparticle surface.⁴⁸ Herein, an R2ab fusion (R2abF) construct was engineered to combine the biofilm-targeting function of R2ab with a nanoparticle-binding protein conjugated to AuNPs via a strong gold-thiol (Ag—S) linkage. For the AuNP-binding protein, a variant of GB3 (a globular protein with 56 amino acid residues) was used. This GB3 variant is known to bind citrate-AuNPs tightly.^{30,49-51} A linker was used to separate R2ab from GB3, reducing the degree to which R2ab interacts with the AuNP surface.³⁰

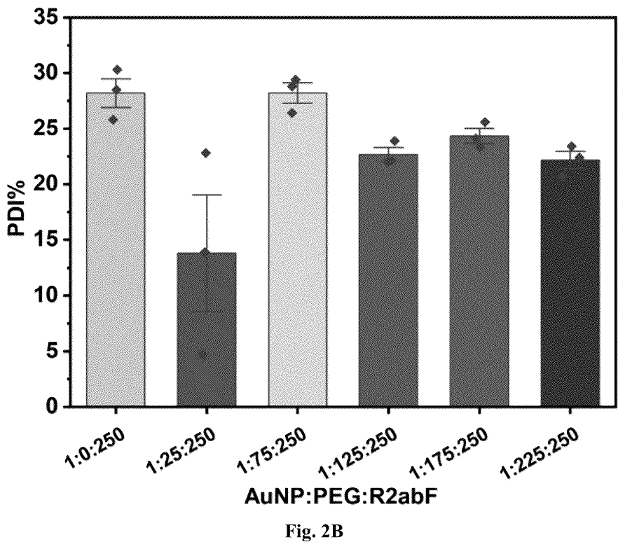

Directly treating 15 nm AuNPs with R2abF was unsuccessful as the particle rapidly aggregated as determined by a large its hydrodynamic diameter (D_{H}; FIG. 2A, gray bars). This was likely due to R2ab binding strongly to the AuNP surface, unfolding and leading to reduced colloidal stability. To control this behavior, thiolated polyethylene glycol (PEG) with a 5,000 average molecular weight (PEG5K) was employed to partially PEGylate the AuNP surface, which increased colloidal stability (FIG. 2A, remaining bars). The polydispersity index (PDI %) and D_{H} were measured for multiple ratios of AuNP, PEG, and R2ab to determine the best formulation of AuNP@PEG5K@R2abF. As the AuNP:PEG5K ratio was increased beyond 1:125, no significant reduction in the D_{H} was observed, and the PDI % remained constant. On this basis, a AuNP:PEG5K:R2abF ratio of 1:125:250 was selected.

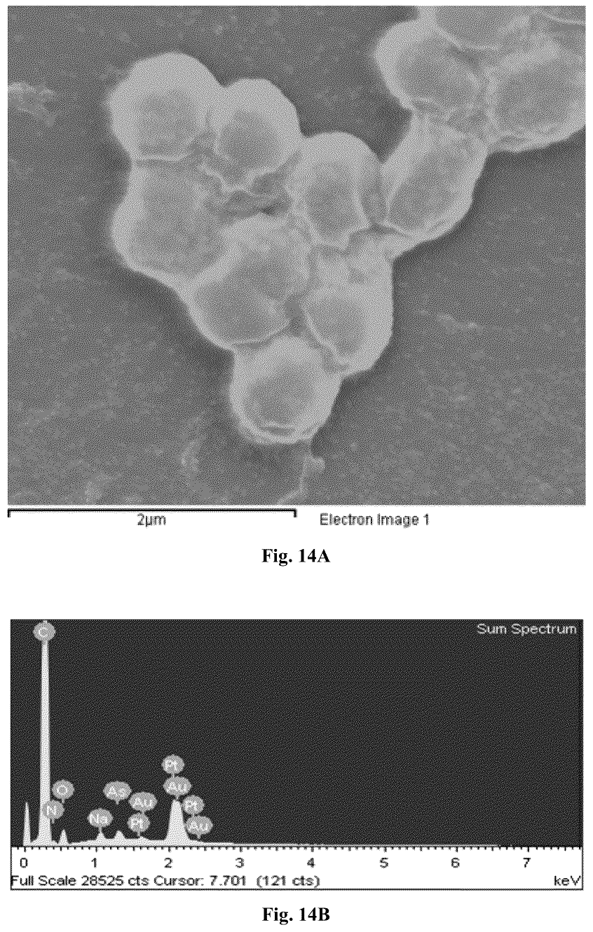

Somarathne et al. have demonstrated that R2ab is efficient at binding to clean polystyrene surfaces.⁴⁴ This property of R2ab was used to confirm that the R2ab is folded, functional, and accessible when incorporated into TRNs. 100 μL of AuNP@PEG5K@R2abF nano-assemblies were added with different AuNP:PEG5K:R2abF ratios, ranging from 1:0:250 to 1:225:250 (FIG. 19). Uniformly stained polystyrene surfaces indicate that the R2ab domain present on all AuNP@PEG5K@R2abF nano-assemblies was able to interact with polystyrene surfaces when PEG5K is present to prevent aggregation. Nano-assemblies containing only PEG do not stain polystyrene surfaces (FIG. 19, control well). Staining was evaluated colorimetrically by evaluating the maximal extinction at 530 nm (FIG. 2C). All the R2abF-containing variants had much greater extinction values than AuNP@PEG5K, demonstrating the R2ab domain is able to target AuNPs to bare polystyrene surfaces but that PEGylation did not (FIG. 2C, green bar).

The current preferred strategy for integrating and improving the functions of synthesized nanomaterials is using multiplex assembly, where multiple functionalization's are employed to enhance particle performance capabilities. Previous work shows that self-assembly or co-assembly of NPs led to functional improvements. For example, Amarasckara et al., Sun et al., and Lin et al. functionalized the AuNP surface with biological or synthetic polymer coatings to enhance the structural ability as well as to improve the photothermal transduction effect of AuNPs in the NIR region.^{25,41,43} Also, it was previous determined that conjugated elastin-like polypeptide (ELP) and a second, inert protein to AuNPs, provided a highly tunable and reversible aggregation transition, leading to photothermal responsiveness.²⁵ Including the R2abF domain and PEG5K lead to a similar response with a high photothermal conversion efficiency (η). At temperatures above Tₜ, the diameter of AuNP@PEG5K@R2abF@ELPA4C increased to several hundred nanometers, as expected for ELP-functionalized particles (FIG. 2D). Below the Tₜ, the particles readily dispersed. Moreover, Tₜ was tunable depending on the AuNP:ELP ratio (Table 1).

Investigations of aggregation reversibility revealed an unexpected trend in the aggregated D_{H}, where the high-temperature D_{H} decreased with each cycle (FIG. 2D, cycle 1 vs. cycle 5). This may be due to structural changes that occur in nano-assemblies throughout cooling and heating cycles. To probe the source of this behavior, a UV-vis extinction spectra was compared between freshly prepared AuNP@PEG5K@R2abF@ELPA4C and samples that had been thermally cycled five times (FIG. 15). No difference was observed in the plasmonic peak, suggesting the trend in D_{H} (FIG. 2D) may originate from protein structural changes as opposed to subtle differences in nanoparticle agglomeration. The lower accuracy of DLS when monitoring large aggregates may have also contributed to this trend.⁵²

Next, was a test to determine if the addition of ELP reduced the ability of R2abF to bind to surfaces. The same colorimetric assay described above was employed with targeted TRN constructs containing AuNP@PEG5K@R2abF@ELPA4C. To start, the construct with the most favorable properties described above in Table 1 was selected and the R2abF: ELPA4C ratio of 1:1 was used, which had a slightly higher Tₜ (39° C.) to physiological temperature. The colorimetric assay demonstrated ELP and R2abF are compatible when functionalized on the same nanoparticle, and the targeted TRN was able to stain polystyrene surfaces more strongly than a control lacking R2abF (FIG. 2E). This result strongly suggests that the R2ab functionality is retained on these engineered nanospheres. The targeted TRNs were further characterized using DLS, TEM, UV-vis, and zeta potential to examine the sequential addition of each component to the AuNP surface. The addition of PEG5K, R2abF, and ELPA4C to AuNPs resulted in an increase in D_{H} (FIG. 2F), but no evidence of aggregation below Tₜ was observed via TEM (FIG. 12). The localized plasmon resonance peak of AuNPs at 520 nm redshifts slightly by 2 nm (FIG. 2G), likely arising from the binding of thiol groups and proteins in the constructs of the present invention.^{25,30} Finally, the zeta potential increased after adding each component (FIG. 2H), corresponding to PEG5K binding, then R2abF, and finally ELPA4C.⁵³ While the lower magnitude of the zeta potential indicates reduced colloidal stability, no issues were observed with unexpected aggregation and the particles could readily be concentrated to 300 nM (3,600 ppm Au).

TABLE 1
Effect on ELP:AuNP ratio on Tₜ of Targeted
TRNs. The AuNP:PEG:R2abF molar ratio
was held constant at 1:125:250.

| | Transition |
|---|---|
| AuNP:ELP | Temperature |
| Ratio | (Tₜ, ° C.) |
| 1:0 | — |
| 1:125 | 42.0 ± 0.1 |
| 1:250 | 39.3 ± 0.6 |
| 1:375 | 39.0 ± 0.1 |
| 1:500 | 38.3 ± 0.6 |
| 1:675 | 38.3 ± 0.6 |

### Colloidal Stability and Cytotoxicity of Targeted TRNs

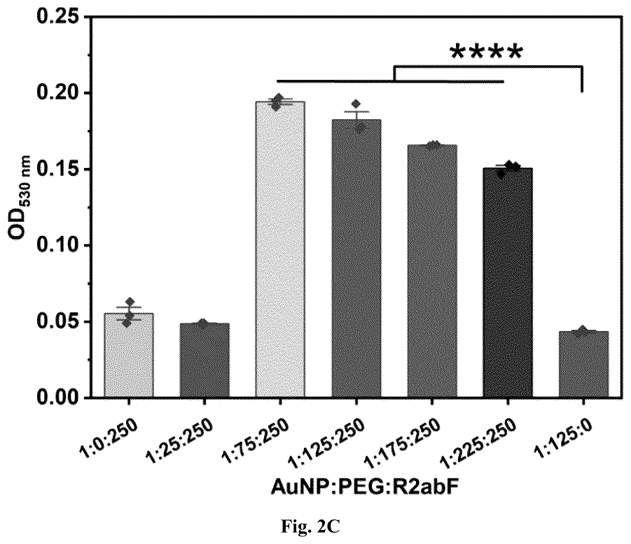

Nanoparticles frequently interact with a variety of biological fluids containing a wide range of proteins and ions that vary significantly from one individual to another. The layer of proteins that forms is called a biological corona, which can interfere with nanoparticle targeting.^{21,54,55} It was determined whether the targeted TRNs interact with blood proteins to form a stable corona that could block biofilm targeting. D_{H} was measured for citrate AuNPs, AuNP@PEG5K, AuNP@GB3NF, AuNP@ELPA4C, AuNP@PEG5K@R2abF, and AuNP@PEG5K@R2abF@ELPA4C with and without 30% fetal bovine serum (FBS, FIG. 3A). A marked corona forms on non-functionalized AuNPs as well as AuNPs functionalized with GB3NF and R2abF alone. However, the addition of ELPA4C passivated the particles, and no significant increase in D_{H} was observed for targeted TRNs. Similar behavior was observed for AuNP@PEG5K and AuNP@ELPA4C nano-assemblies. Thus, while R2abF and GB3NF alone appear to drive corona formation, the inclusion of PEG5K and ELP counteracts this behavior, giving targeted TRNs good surface passivation thereby reducing corona formation.^{56,57}

Next, the targeted TRNs were tested to see if they affected the viability of S. epidermidis bacterial cells. Previously, it was determined that TRNs lacking R2ab did not affect bacterial growth rates, nor did they alter the viability of human HEK-293 cells grown in cell culture.²⁵ As expected, the targeted TRNs containing R2ab did not alter the previous results. A final AuNP concentration of 20 nM (240 ppm total Au) was added to a fresh culture of S. epidermidis, and the cell growth curve was monitored using a plate reader (FIG. 3B). All the nano-assemblies used in the study showed no change in cell growth compared to the control, confirming that all the nano-assemblies (including targeted TRNs) were biocompatible at an Au concentration of 240 ppm after incubation for 24 hours. This is consistent with prior studies of functionalized AuNPs; when compounds such as cetyl-trimethylammonium bromide (CTAB) were avoided, functionalized AuNPs were generally biocompatible.⁵⁸⁻⁶⁰ Even at 40 nM (480 ppm Au), no observable effect on bacterial growth was observed for any of the tested constructs (FIG. 3C). Thus, in the absence of NIR irradiation, the nano-assemblies used in this study resist corona formation and do not adversely affect cell function.

### Assessing Selective Biofilm Adherence of Targeted TRNs

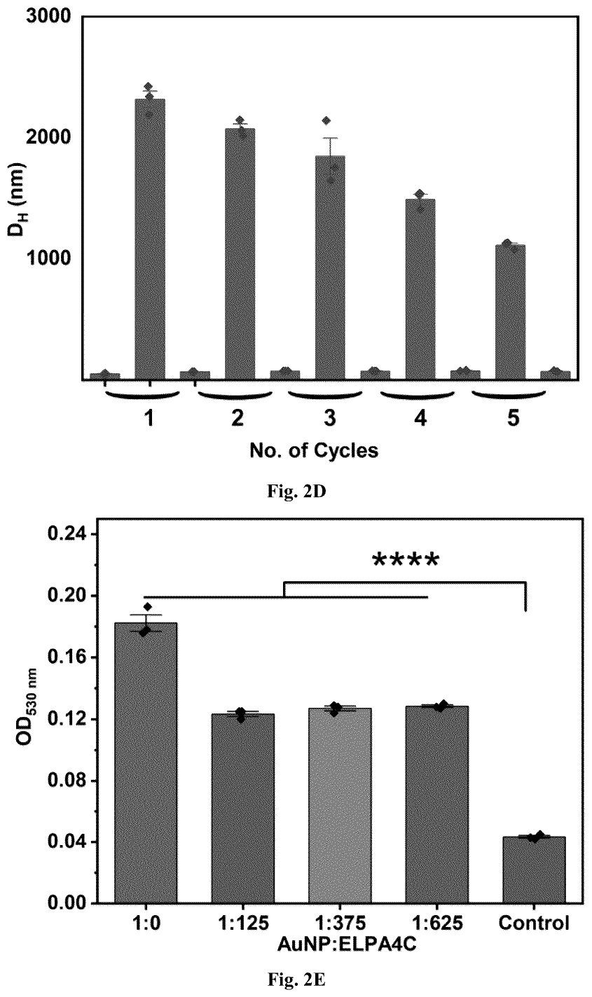

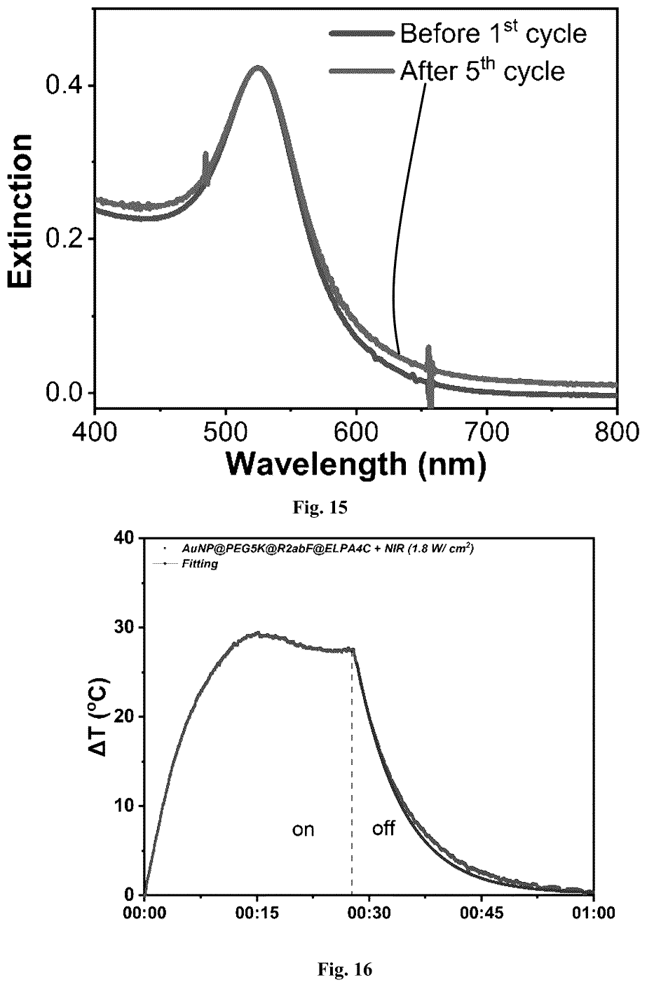

The effectiveness of bacterial targeting was assessed using the engineered R2ab fusion domain. R2ab targets the cell wall components of S. epidermidis,²⁸ and it was expected that the R2ab function would localize the targeted TRNs to S. epidermidis biofilms, enhancing their effect. The interaction was first tested with biofilms grown statically in a 96-well plate. The biofilms were treated with 20 nM (240 Au ppm) of AuNP@PEG5K@R2abF@ELPA4 or AuNP@PEG5K as a control (see Materials and Methods). The biofilms were washed three times to remove excess TRNs, and the remaining nanoparticles were measured using extinction at 530 nm (FIG. 20). The retention of all targeted TRNs (regardless of ELP content) was significantly higher than the AuNPs coated with PEG alone. To assess binding quantitatively, biofilms were digested in aqua regia, and the total content was measured using inductively coupled plasma-mass spectrometry (ICP-MS, FIG. 4A). Of the six systems tested, only targeted TRNs containing ELP, PEG5K, and R2abF showed the desired specificity, where adhesion to biofilms was higher than to serum-coated surfaces. AuNPs lacking ELP (but containing R2ab) had the opposite effect, where serum-coated polystyrene bound more. This may result from the anti-fouling properties provided by ELP (FIG. 3A), and it may also result from incomplete coverage of polystyrene by the 30% FBS. For all the other particles tested, binding to serum-coated surfaces was higher than binding to biofilms, and the lack of functionalization on citrate AuNPs led to the highest binding of all (FIG. 4A, orange bars). Thus, targeted TRNs containing PEG5K, ELP, and R2abF exhibited specific binding toward S. epidermidis biofilms as designed.

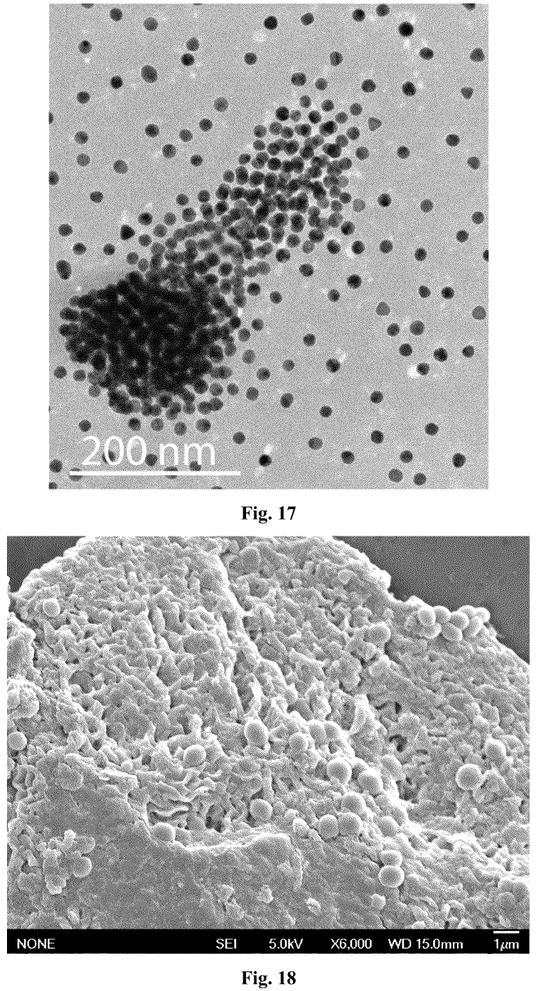

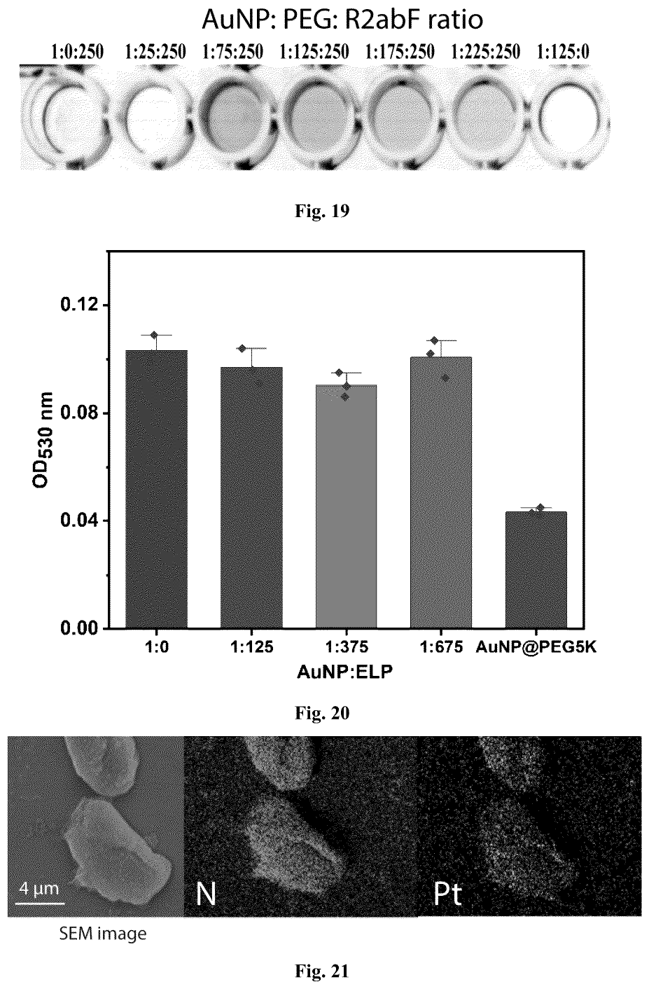

To visualize specific binding, scanning electron microscopy (SEM) coupled with energy dispersive X-ray spectroscopy (EDS) was employed to monitor the presence of targeted TRNs (but not AuNPs@PEG5K) on biofilm surfaces (FIGS. 13 and 14). The characteristic peak at 2 keV confirmed the presence of gold on the biofilm surface when treated with targeted TRNs; the corresponding peak had a much lower relative intensity when biofilms were treated with PEGylated AuNPs alone. Visually, the Au signals from EDS appear localized to regions where biofilm is present and not in the polystyrene background (FIG. 4B and FIG. 21). Thus, optical staining, quantitative measurement by ICP-MS, and electron microscopy all support the conclusion that targeted TRNs containing the S. epidermidis R2ab domain were able to selectively bind S. epidermidis biofilms, whereas nanoparticles lacking R2ab did not selectively bind the biofilms.

### In Vitro Antibiofilm Activity of Targeted TRNs Under NIR Irradiation

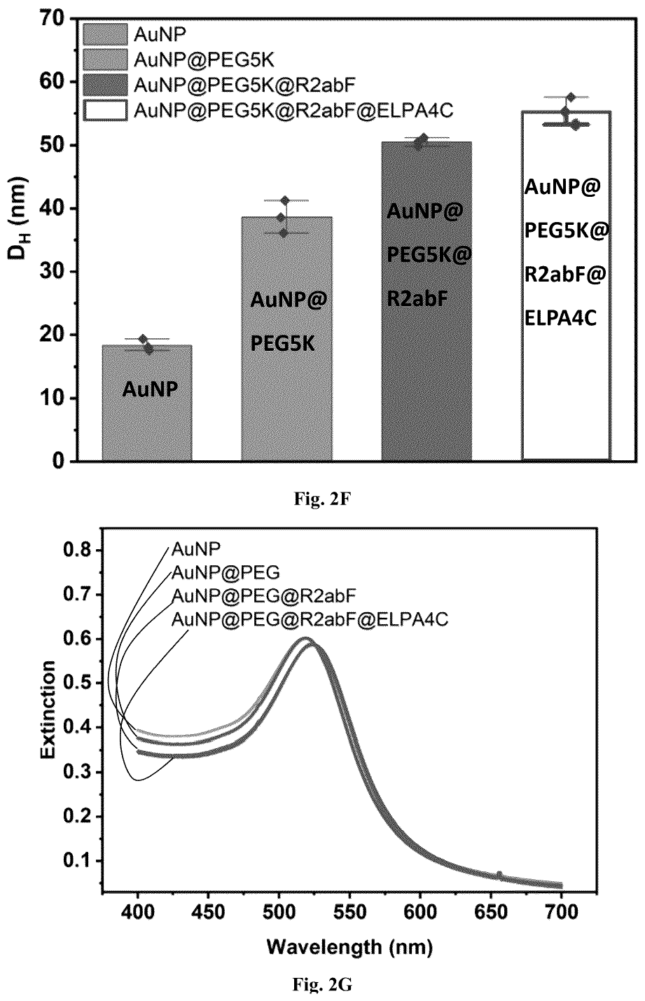

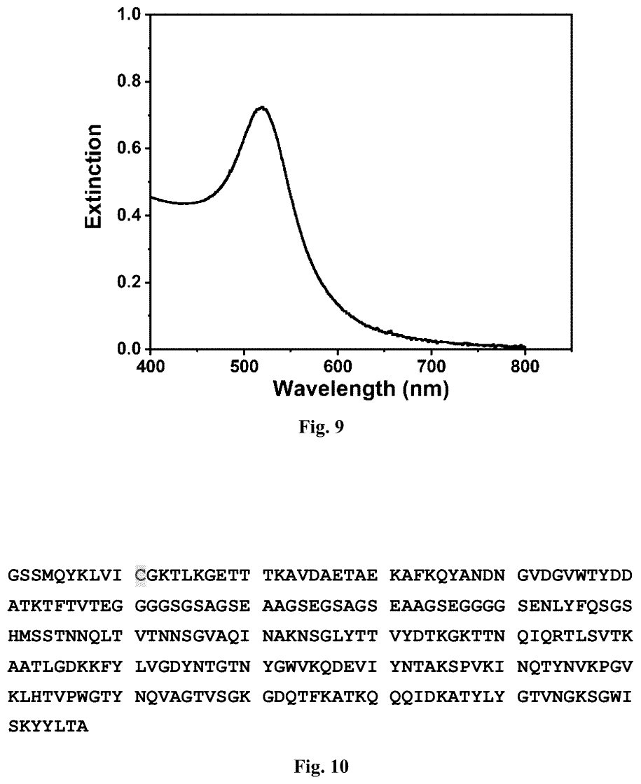

The effectiveness of biofilm-targeted TRNs was assessed in its ability to eradicate biofilm bacteria upon exposure to NIR irradiation. Solutions of targeted TRNs were added to S. epidermidis biofilm-coated polystyrene surfaces and then exposed to NIR light to assess the bactericidal impact. Constructs lacking ELP and R2abF were used as controls. After being exposed to NIR light for the first five minutes, the S. epidermidis biofilm containing AuNPs all showed an increase in the temperature (FIG. 5A-C). However, at or above 20 nM particle concentration (240 ppm of Au), the targeted TRNs outperformed the temperature increase of the controls. At 30 nM (360 ppm of Au), a maximum temperature of around 60° C. was obtained, which is sufficient to kill bacteria embedded in biofilms.²⁵ The photothermal conversion efficiency (η) of AuNP@PEG5K@R2abF@ELPA4C was calculated to be around 40±2% (FIG. 16), which was higher than that of AuNP@PEG5K (31±5%) as reported previously.²⁵ After treatment with targeted TRNs and exposure to NIR light for 5 minutes, the number of surviving bacteria dramatically decreased (FIGS. 5D and 5E), demonstrating an enhanced photothermal bactericidal effect that was absent for TRNs lacking ELP. Targeted TRNs aggregate reversibly at elevated temperatures, which leads to this enhanced photothermal effect (FIG. 17).

### Assessing the Anti-Biofilm Effectiveness of Targeted TRNs Under Continuous Flow Conditions

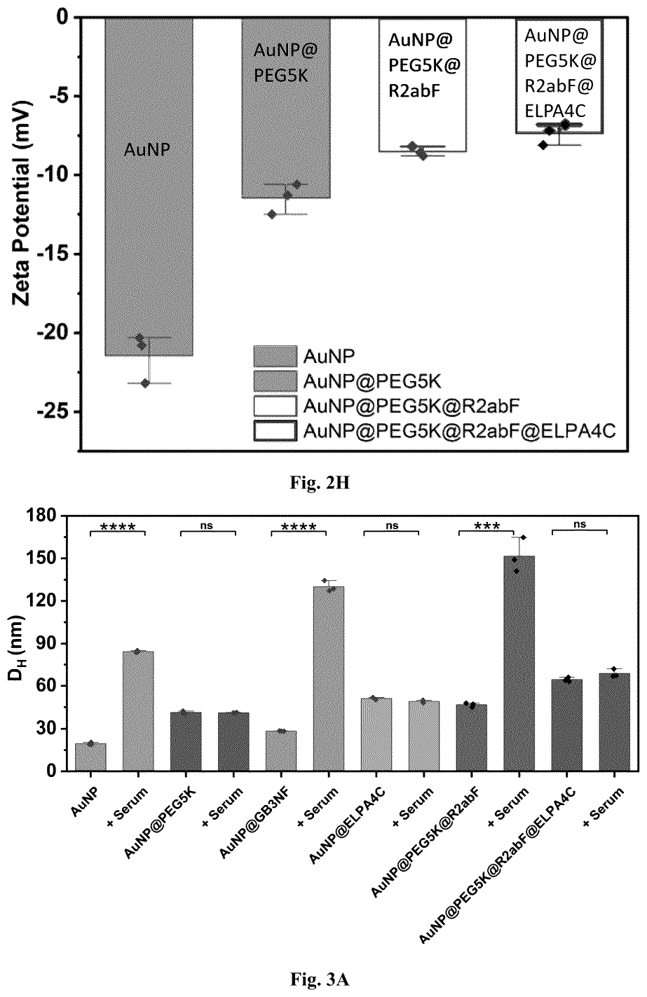

The experiments above demonstrated that targeted TRNs were as effective when the targeting domain was included.²⁵ The additional PEG and R2abF functionalities only marginally impacted measurements of photothermal conversion efficiency. It was then tested to determine whether the targeting domain is sufficient to localize TRNs to biofilms in the presence of continuous flow. These conditions were like conditions present in in vivo systems. An inexpensive dynamic biofilm system was developed to study nanoparticle adhesion properties under continuous flow through polystyrene tubing derived from a serological pipet. A 3D printed base was employed (FIGS. 6A and 6B) to hold 3 tubes in series. A 12 V peristaltic pump was used to drive a flow of 5 mL min⁻¹ (linear rate of 4.6 cm s⁻¹) of BHI medium and targeted TRNs through the tubes. For the 1.5 mm inner diameter tubes used here, this rate was comparable to a rate that would be experienced in the arteries of the circulatory system.^{27,61,62} This system is similar to the Modified Robbins Device (MRD) discussed by Coenye and Nelis.⁶³ Polytetrafluoroethylene (PTFE) tubing was used to circulate the solution, as biofilm growth was significantly reduced on PTFE surfaces. New PTFE tubing and polystyrene tubes were used after each experiment to maintain consistency between experiments.

By using this platform to conduct 48 hour dynamic biofilm tests with S. epidermidis, biofilm formation was detected on the inner walls of the plastic tubes (FIG. 6C). Crystal violet staining (FIG. 6D) and SEM imaging (Supplementary Material, FIG. S12) were used to confirm the presence of the biofilm. The excess bacteria on the inner walls of the plastic tubes were removed using PBS buffer. AuNPs, AuNP@PEG5K, and AuNP@PEG5K@R2abF@ELPA4C (30 nM or 360 Au ppm) were sent through the system to individually assess the biofilm adhesion on the plastic tubes. The results were consistent with the experiments performed under static conditions (FIG. 4A). No biofilm binding was detected for AuNP@PEG5K particles, but the addition of the R2ab fusion domain effectively stained the biofilm red (FIG. 6E). The complete targeted TRN particle (AuNP@PEG5K@R2abF@ELPA4C) also stained biofilms (FIG. 6F), demonstrating its ability to adhere to the biofilm even under dynamic conditions.

Next, the photothermal properties of the biofilm-bound nano-assemblies were determined using an 808 nm NIR laser. A 3D-printed structural mount was used to hold biofilm-coated tubes during laser irradiation (FIG. 6G). After treatment with nanoparticles, each tube was cut into three 1 cm sections. These sections, containing biofilm only, biofilm and AuNP@PEG5K, or biofilm and targeted TRNs (AuNP@PEG5K@R2abF@ELPA4C), were irradiated with a 1.8 W cm⁻² 808 nm NIR laser for 15 minutes to observe their temperature response (FIG. 6H). A longer laser treatment time was needed to reach a steady temperature, presumably due to the decreased concentration of TRNs. However, a significant temperature increase was obtained for targeted TRNs (FIG. 6I), where the final temperature increase was nearly 13° C. more than with AuNP@PEG5K (red vs. green curve). To assess the bactericidal effect, each 1 cm section was soaked in 1 mL of PBS media with agitation for 30 minutes, allowing the live cells to detach from the biofilm. The CFU mL⁻¹ of this solution was measured using the serial dilution assay. Significantly fewer cells grew from tubes treated with targeted TRNs and NIR irradiation than the untreated control or tubes treated with AuNP@PEG5K and NIR irradiation (FIG. 6J); i.e. an almost 104-fold reduction in CFU mL⁻¹ was observed. This was consistent with bacteria grown in static biofilms (FIG. 5E). Thus, the targeted TRNs of the invention were shown to effectively target photothermally active nanoparticles to S. epidermis biofilms, both under static and dynamic flow conditions. This demonstrates that bacterial proteins such as R2ab, which bind cell wall components, can be used to functionalize nanomaterials, effectively targeting those nanomaterials to infection sites.

Recently, it was shown that nanoparticles functionalized with Hoechst dye could be used to target nanoparticles to biofilms, since this dye can form a specific adduct with extracellular DNA in the biofilm.⁶⁴ Herein, the present invention employs a similar strategy with the bacteria's own extracellular protein. Using the S. epidermidis R2ab domain, nanoparticles can be used to target S. epidermidis biofilms. The mechanism is driven by R2ab's natural ability to bind cell wall components, which are not expected to be present in mammalian tissues. These targeted TRNs (AuNP@PEG5K@R2abF@ELPA4C) agglomerated in response to elevated temperature, forming particle clusters with high photothermal conversion efficiency upon near-infrared radiation. Effective and selective bactericidal properties were confirmed for both statically grown biofilms and biofilms grown and treated under continuous flow conditions. The temperature responsiveness and selective adhesion of the synthesized particles may be employed for developing intelligent photothermal transducers for thermal ablation of S. epidermidis biofilms. The targeted TRNs of the invention provide a simple and generalizable strategy for efficiently making targeted agents for photothermal therapy.

SEQUENCES
R2ab protein
SEQ ID NO: 1
MSSTNNQLTV TNNSGVAQIN AKNSGLYTTV YDTKGKTTNQ IQRTLSVTKA ATLGDKKFYL
VGDYNTGTNY GWVKQDEVIY NTAKSPVKIN QTYNVKPGVK LHTVPWGTYN QVAGTVSGKG
DQTFKATKQQ QIDKATYLYG TVNGKSGWIS KYYLTA
R2ab fusion protein construct
SEQ ID NO: 2
CCATGGGCAGCCATCACCATCACCATCACAGCAGCGGCTTAGTGCCACGAGGGTCTAGCATG
CAGTACAAATTAGTTATCTGTGGTAAAACATTGAAAGGCGAAACAACTACTAAAGCTGTT
GATGCTGAAACTGCAGAAAAAGCTTTCAAACAATACGCTAACGACAACGGTGTTGACGGT
GTTTGGACTTACGACGATGCGACTAAGACCTTTACAGTTACTGAAGGAGGTGGCGGTAGC
GGTAGCGCAGGTAGCGAAGCAGCAGGTTCAGAAGGTTCAGCTGGCTCAGAAGCAGCTGGC
AGCGAAGGTGGTGGTGGTAGTGAAAATTTGTACTTCCAATCCGGGTCTCATATGAGCAGC
ACCAATAATCAGCTGACCGTTACCAATAATAGCGGTGTTGCACAGATTAATGCGAAAAAT
AGCGGTCTGTATACCACCGTGTATGATACCAAAGGTAAAACCACCAATCAGATTCAGCGT
ACCCTGAGCGTGACCAAAGCAGCAACCCTGGGTGATAAAAAGTTTTATCTGGTGGGTGAT
TATAACACCGGCACCAATTATGGTTGGGTTAAACAGGATGAAGTGATTTACAATACCGCA
AAAAGTCCGGTGAAAATCAACCAGACCTATAATGTTAAACCGGGTGTTAAACTGCATACC
GTTCCGTGGGGTACATATAATCAGGTTGCAGGCACCGTTAGCGGTAAAGGTGATCAGACC
TTTAAAGCAACCAAACAGCAGCAGATTGATAAAGCCACCTATCTGTATGGTACGGTGAAT
GGTAAAAGCGGTTGGATCAGCAAATATTACCTGACCGCATAAGGATCC
(DNA)
Modified third IgG binding domain from streptococcal protein G (GB3)
SEQ ID NO: 3
MQYKLVICGKTLKGETTTKAVDAETAEKAFKQYANDNGVDGVWTYDDATKTFTVTE
Ubiquitin protein sequence
SEQ ID NO: 4
MQIFVKTLTG KTITLEVEPS DTIENVKAKI QDKEGIPPDQ QRLIFAGKQL
EDGRTLSDYNIQKESTLHLV LRLRGG
Pin1 WW domain protein sequence
SEQ ID NO: 5
MGSSHHHHHH SSGLVPRGSH GMADEEKLPP GWEKRMSRSS GRVYYFNHIT
NASQWERPSG NS
3FN3 domain protein sequence
SEQ ID NO: 6
MGSSHHHHHHSSGLVPRGSHMGTTTAPDAPPDPTVDQVDDTSIVVRWSRPQAPITGYRIVYSPSV
EGSSTELNLPETANSVTLSDLQPGVQYNITIYAVEENQESTPVVIQQETTGTPRSDGT
Linker
SEQ ID NO: 7
GGGGSGSAGS EAAGSEGSAG SEAAGSEGGG GSE
Elastin-like polypeptide of A4C
SEQ ID NO: 8
VPGCG-(VPGVG)₇-VPGTG-(VPGVG)₇-VPGAG-(VPGVG)₇-VPGTG-(VPGVG)₇-VPGAG-(VPGVG)₇-
VPGTG
Elastin-like polypeptide of A84C
SEQ ID NO: 9
VPGAG-(VPGVG)₇-VPGTG-(VPGVG)₇-VPGCG-(VPGVG)₇-VPGTG-(VPGVG)₇-VPGAG-(VPGVG)₇-
VPGTG
R2ab fusion protein construct
SEQ NO: 10:
MGSHHHHHHS GL VPR
GSSMQYKLVI CGKTLKGETT TKAVDAETAE KAFKQYANDN GVDGVWTYDD
ATKTFTVTEG GGGSGSAGSE AAGSEGSAGS EAAGSEGGGG SENLYFQSGS
HMSSTNNQLT VTNNSGVAQI NAKNSGLYTT VYDTKGKTTN QIQRTLSVTK
AATLGDKKFY LVGDYNTGTN YGWVKQDEVI YNTAKSPVKI NQTYNVKPGV
KLHTVPWGTY NQVAGTVSGK GDQTFKATKQ QQIDKATYLY GTVNGKSGWI
SKYYLTA GS
(PROTEIN)
R2ab fusion protein construct
SEQ ID NO: 11
GSSMQYKLVICGKTLKGETTTKAVDAETAEKAFKQYANDNGVDGVWTYDD
ATKTFTVTEGGGGSGSAGSEAAGSEGSAGSEAAGSEGGGGSENLYFQSGSHMSSTNNQLT
VTNNSGVAQINAKNSGLYTTVYDTKGKTTNQIQRTLSVTKAATLGDKKFY
LVGDYNTGTNYGWVKQDEVIYNTAKSPVKINQTYNVKPGVKLHTVPWGTY
NQVAGTVSGKGDQTFKATKQQQIDKATYLYGTVNGKSGWISKYYLTA
R2ab fusion protein construct
SEQ ID NO: 12
MGSHHHHHHSGLVPRGSSMQYKLVICGKTLKGETTTKAVDAETAEKAFKQYANDNGVDGVWT
YDDATKTFTVTEGGGGSGSAGSEAAGSEGSAGSEAAGSEGGGGSENLYFQSGSHMSSTNNQLTV
TNNSGVAQINAKNSGLYTTVYDTKGKTTNQIQRTLSVTKAATLGDKKFYLVGDYNTGTNYGW
VKQDEVIYNTAKSPVKINQTYNVKPGVKLHTVPWGTYNQVAGTVSGKGDQTFKATKQQQIDK
ATYLYGTVNGKSGWISKYYLTA

What is claimed is:

1. A thermally responsive nanosphere for targeted binding to biofilms, wherein the nanosphere comprises:
a gold nanoparticle core functionalized with:
a) a polyethylene glycol;
b) an R2ab fusion protein construct including an anchor, a linker and a S. epidermidis R2ab protein having SEQ ID NO. 1,
wherein the anchor is any stable globular domain and comprises a surface accessible cysteine residue for attachment to the gold nanoparticle core; and
c) an elastin-like polypeptide.

2. The thermally responsive nanosphere of claim 1, wherein the R2ab fusion protein construct has SEQ ID NO. 2.

3. The thermally responsive nanosphere of claim 1, wherein the elastin-like polypeptide is selected from the group consisting of polypeptides having SEQ ID NO. 8 and SEQ ID NO. 9.

4. The thermally responsive nanosphere of claim 2, wherein the elastin-like polypeptide is selected from the group consisting of polypeptides having SEQ ID NO. 8 and SEQ ID NO. 9.

5. The thermally responsive nanosphere of claim 1, wherein the polyethylene glycol is thiolated, and optionally, the polyethylene glycol has an average molecular weight of from about 1,000 g/mol to about 10,000 g/mol.

6. The thermally responsive nanosphere of claim 2, wherein the polyethylene glycol is thiolated, and optionally, the polyethylene glycol has an average molecular weight of from about 1,000 g/mol to about 10,000 g/mol.

7. The thermally responsive nanosphere of claim 3, wherein the polyethylene glycol is thiolated, and optionally, the polyethylene glycol has an average molecular weight of from about 1,000 g/mol to about 10,000 g/mol.

8. The thermally responsive nanosphere of claim 4, wherein the polyethylene glycol is thiolated, and optionally, the polyethylene glycol has an average molecular weight of from about 1,000 g/mol to about 10,000 g/mol.

9. The thermally responsive nanosphere of claim 1, wherein the linker is a protein having SEQ ID NO. 7.

10. The thermally responsive nanosphere of claim 1, wherein the anchor is selected from the group consisting of a modified third IgG binding domain from streptococcal protein G (GB3) having SEQ ID NO. 3, a ubiquitin protein having SEQ ID NO. 4, a Pin1 WW domain having SEQ ID NO. 5, and a fibronectin protein domain 3FN3 having SEQ ID NO. 6.

11. The thermally responsive nanosphere of claim 1, wherein the anchor binds to the gold nanoparticle core.

12. The thermally responsive nanosphere of claim 11, wherein a molar ratio of the gold nanoparticle core to the elastin-like polypeptide is from about 1:25 to about 1:675.

13. The thermally responsive nanosphere of claim 11, wherein a molar ratio of the gold nanoparticle core to the elastin-like polypeptide is from about 1:125 to about 1:500.

14. The thermally responsive nanosphere of claim 1, wherein a molar ratio of the gold nanoparticle core to the polyethylene glycol to the R2ab fusion protein construct is from about 1:25:250 to 1:225:250.

15. The thermally responsive nanosphere of claim 1, wherein a molar ratio of the gold nanoparticle core to the polyethylene glycol to the R2ab fusion protein construct is from about 1:75:250 to about 1:225:250.

16. The thermally responsive nanosphere of claim 1, wherein the gold nanoparticle has a hydrodynamic diameter of from about 5 nm to 50 nm, as determined by dynamic light scattering.

17. The thermally responsive nanosphere of claim 1, wherein the gold nanoparticle has a hydrodynamic diameter of from about 10 nm to 30 nm, as determined by dynamic light scattering.

18. The thermally responsive nanosphere of claim 1, wherein the gold nanoparticle has a hydrodynamic diameter of about 15 nm, as determined by dynamic light scattering.

19. The thermally responsive nanosphere of claim 1, wherein the R2ab domain is fused to a C-terminal end of the linker.

20. A method for treating a biofilm containing S. epidermidis bacteria comprising binding a plurality of the nanospheres of claim 1 to the biofilm and exposing the biofilm with the bound nanoparticles to laser irradiation.
